(19) United States
(12) Patent Application Publication
(10) Pub. No.: US 2015/0245882 A1
(43) Pub. Date: Sep. 3, 2015

(54) SYSTEMS FOR LINEAR MAPPING OF LUMENS

(71) Applicant: Angiometrix Corporation, Bethesda, MD (US)

(72) Inventors: Vikram VENKATRAGHAVAN, Bangalore (IN); Raghavan SUBRAMANIYAN, Bangalore (IN)

(73) Assignee: Angiometrix Corporation, Bethesda, MD (US)

(21) Appl. No.: 14/535,204

(22) Filed: Nov. 6, 2014

Related U.S. Application Data
(63) Continuation of application No. PCT/US2013/039995, filed on May 7, 2013.
(60) Provisional application No. 61/644,329, filed on May 8, 2012.
(60) Provisional application No. 61/763,275, filed on Feb. 11, 2013.

(51) Int. Cl. A61B 19/00; A61B 1/00; A61M 5/00; A61M 25/10; A61M 25/09

(52) U.S. Cl. CPC A61B 19/54; A61M 25/10; A61M 25/09; A61M 5/007; A61B 19/5244; A61B 1/00009; A61M 2025/09166; A61B 2019/5291; A61B 2019/5251; A61B 2019/5287; A61B 2019/5265; A61B 2019/5238; A61B 2019/5236; A61B 2019/5276; A61B 2019/5231; A61B 2019/5295; A61M 2025/1079; A61B 2019/5466; A61B 2019/5454; A61B 2019/5479; A61B 2019/5425

(57) ABSTRACT

Systems for linear mapping of lumens are described which utilizes methods to create a linearized view of a lumen using multiple imaged frames. In reality a lumen has a trajectory in 3-D, but only a 2-D projected view is available for viewing. The linearized view unravels this 3-D trajectory thus creating a linearized map for every point on the lumen trajectory as seen on the 2-D display. In one mode of the invention, the trajectory is represented as a linearized display along 1 dimension. This linearized view is also combined with lumen measurement data and the result is displayed concurrently on a single image. In another mode of the invention, the position of a treatment device is displayed on the linearized map in real time. In a further extension of this mode, the profile of the lumen dimension is also displayed on this linearized map.

### CROSS-REFERENCE TO RELATED APPLICATIONS

This application is a continuation of International Application No. PCT/US2013/039995 filed May 7, 2013, which claims the benefit of priority to U.S. Provisional Application No. 61/644,329 filed May 8, 2012 and 61/763,275 filed Feb. 11, 2013, each of which is incorporated herein by reference in its entirety.

### FIELD OF THE INVENTION

The invention relates generally to intravascular medical devices. More particularly, the invention relates to guidewires, catheters, and related devices which are introduced intravascularly and utilized for obtaining various physiological parameters and processing them for mapping of various lumens.

### BACKGROUND OF THE INVENTION

There are several devices such as IVUS and OCT wires or catheters that measure dimensions of lumens. These devices are inserted into the lumen to the end of or just past the region of interest. The device is then pulled back using a stepper motor while lumen measurements are made. This allows for creating a “linear” map of lumen dimension along the lumen. In a typical representation, the X axis of the map would be the distance of the measurement point from a reference point, and the Y axis would be the corresponding lumen dimension (e.g. cross sectional area or diameter). This allows the physician to ascertain the length, cross-sectional area and profile of a lesion (diseased portion of a blood vessel). Both, the length and cross-sectional area of a lesion are desirable to determine the severity of the lesion as well as the potential treatment plan. For example, if a stent is to be deployed, the diameter of the stent is determined by the measured diameter in the neighboring un-diseased portion of the blood vessel. The length of the stent would be determined by the length of significantly diseased section of the blood vessel.

While IVUS and OCT give a good estimate of the length and cross-sectional area of a lesion, one problem is that when the treatment is delivered it does not preserve position. After measurement is made, the measurement device is retracted and the treatment device is introduced into the lumen. There is no existing mechanism to determine if the stent is positioned correctly at the diseased site. The “linear” map created during measurement is available, but the current position of the stent in this linear map is not available. In other words the obtained information is not co-registered with the X-ray image.

The other problem is that the primary display used by physicians to view the X-ray images during diagnosis and treatment is typically a 2-D image taken from a certain angle and with a certain zoom factor. Since these images are a projection of a structure that is essentially 3-D in nature, the apparent length and trajectory would be a distorted version of the truth. For example, a segment of a blood vessel that is 20 mm, the apparent length of the segment depends on the viewing angle. If the segment is in the image plane, it would appear to be of a certain length. If it subtends an angle to the image plane, it appears shorter. This makes it difficult to accurately judge actual lengths from an X-ray image. Moreover, in a quasi-periodic motion of a moving organ, different phases during the motion correspond to different structure of lumen in 3-D. This in turn corresponds to different 2-D projections in each phase of the quasi-periodic motion. Thus creating a linearized and co-registered map of a lumen can either be done for each phase of the motion separately or it can be done for a chosen representative phase. In case of the latter, mapping of the 2-D projection of lumen from each individual phase to the representative phase is needed. Even in this latter case, it is not practically possible to avoid the need for motion compensation even if the 2-D projection from a chosen phase of the heartbeat is used. There are several reasons for this. Firstly, the contribution to motion also comes from other reasons such as breathing. This motion also needs to be accounted for. Secondly, because images are captured at discrete points in time (e.g., at 15 frames per second), there may not be a frame available at precise time instance of a particular phase of the heartbeat. Choosing the nearest frame would leave behind a residual motion that can be significant and would need to be compensated for. Thirdly, choosing only one phase of the heartbeat causes a large time gap between two successive frames chosen for a particular phase of the heartbeat. For example, if the heart rate is 60 beats per minute, only one frame per second would be used for processing (typically images are available at 15 or 30 frames per second). This would make it very difficult to track moving markers on the device.

Accordingly, a system that allows co-registering of measured lumen dimensions with a position of the treatment device and details about how the linear map is created with multiple imaged frames and further details about co-registrations is desired.

### SUMMARY OF THE INVENTION

Disclosed are efficient methods to create a linearized view of a body lumen with the help of multiple image frames. In reality a lumen has a trajectory in 3-D, but only a 2-D projected view is available for viewing. The linearized view unravels this 3-D trajectory thus creating a linearized map for every point on the lumen trajectory as seen on the 2-D display. In one mode of the invention, the trajectory is represented as a linearized display along 1-dimension. This linearized view is also combined with lumen measurement data and the result is displayed concurrently on a single image referred to as the analysis mode. This mode of operation can assist an interventionalist in uniquely demarcating a lesion, if there are any, and identify its position. Analysis mode of operation also helps in linearizing the blood vessel while an endo-lumen device is inserted in it (or manually pulled back) as opposed to the popularly used technique of motorized pullback. In another mode of the invention, the position of a treatment device is displayed on the linearized map in real time referred to as the guidance mode. Additionally, the profile of the lumen dimension is also displayed on this linearized map.

Examples of devices and methods for obtaining various dimensions of lumens and which may be used with the devices, systems, and methods disclosed herein may be seen in further detail in the following: U.S. Prov. 61/383,744 filed Sep. 17, 2010; U.S. application Ser. No. 13/159,298 filed Jun. 13, 2011 (U.S. Pub. 2011/0306867); Ser. No. 13/305,610 filed Nov. 28, 2011 (U.S. Pub. 2012/0101355); Ser. No. 13/305,674 filed Nov. 28, 2011 (U.S. Pub. 2012/0101369); Ser. No. 13/305,630 filed Nov. 28, 2011 (U.S. Pub. 2012/0071782); and PCT/US2012/034557 filed Apr. 20, 2012 (designating the U.S.). Each of these applications is incorporated herein by reference in its entirety and for any purpose.

Other aspects of the invention deal with reducing the complexity of image processing that enables a real time implementation of the algorithm. In one aspect, the trajectory of an endo-lumen device is determined, and future frames use a predicted position of the device to narrow down the search range. Detection of endo lumen device and detection of radiopaque markers are combined to yield a more robust detection of each of the components and results in a more accurate linearized map. The method used to compensate for the motion of the moving organ by identifying the endo-lumen device in different phases of the motion is novel. This motion compensation in turn helps in generating a linearized and co-registered map of lumen in a representative phase of the quasi-periodic motion and in further propagating the generated map to other phases of the motion. First the two ends of the visible segment of the endo lumen device—for e.g. in a guide-wire the tip of the guide catheter and the distal coil of the guidewire—are detected. Subsequently different portions of the endo-lumen device are detected along with any radiopaque markers that may be attached to it. Another novel aspect of the invention is the mapping of the detected endo lumen segment from any or all of the previous frames to the current frame to reduce the complexity in detecting the device in subsequent frames. The detection of the endo lumen device itself is based on first detecting all possible tube like structures in the search region, and then selecting and connecting together a sub-set of such structures based on smoothness constraints to reconstruct the endo lumen device. Further, prominent structures on the guidewire are detected more reliably and are given higher weight when selecting the subset of structures. In another variant of this invention, only a subset of the endo lumen segment is detected. This is done in an incremental fashion and only the region relevant for the treatment can be detected and linearized.

Another aspect of the invention is to compensate for motion due to heartbeat and breathing, camera angle change or physical motion of the patient or the platform. The linearized view is robust to any of the aforementioned motion. This is done by using prominent structures or landmarks along the longitudinal direction of the lumen e.g. tip of the guide catheter, distal coil in a deep-seated guide wire section, stationary portion of the delineated guide-wire, stationary radiopaque markers or the farthest position of a moving radiopaque marker along the lumen under consideration, anatomical landmark such as branches along the artery. The linearized map is made with reference to these points.

Other aspects of the invention deal with reducing the complexity of image processing algorithm that enables a real time implementation of the algorithm and compensating for the periodic motion of the organ.

The image processing aspects of the innovation deals with the following:
1. Tapping the live feed video, ECG and other vital signs from the output of the imaging device.
2. Automatic selection of frames to process along with their region of interest.
3. Tracking of the endo-lumen device even in cases where orientation, position and magnification of the imaging device are altered during the procedure.
4. Quantification of biological properties of the vessel such as vessel compliance—for e.g. movement of various parts of the artery—at the time of heartbeat during a cardiac intervention and vessel tortuosity (twists and turns in a vessel)
5. Selection of lesion delineators.
6. Motion compensation of the endo lumen device for computing the linearized map.
7. Selection of the frames where artery is being highlighted by an injected dye and using these frames for analyzing the variation of artery diameter.
8. Automatic blood vessel diameter measurement—also known as QCA.

### BRIEF DESCRIPTION OF THE DRAWINGS

FIG. 1 shows mapping from a 2-D curve to linear representation.

FIG. 2 shows a guidewire and catheter with markers and/or Electrodes.

FIG. 3 shows a guidewire or catheter placed in a curved trajectory.

FIG. 4 shows a guide catheter and guidewire used in angioplasty.

FIG. 5 shows an illustration of the radiopaque electrodes along a guidewire inside a coronary artery.

FIG. 6 shows a block diagram illustrating various steps involved in construction of a linear map of the artery.

FIG. 7 shows a variation of the co-ordinates of the electrodes due to heart-beat.

FIG. 8 shows detection of distal coil.

FIG. 9A shows detected ends of the guidewire.

FIG. 9B shows a variation of the correlation score in the search space.

FIG. 10 shows an example of tube likeliness.

FIG. 11 shows a guidewire mapped from previous frame.

FIG. 12 show guidewire identification and refinement.

FIG. 13 shows a detected guidewire after spline fit.

FIG. 14 shows a graph indicating detection of maxima in the tube-likeliness plot taking the inherent structure of markers into consideration.

FIG. 15 shows radiopaque markers detected.

FIG. 16 shows illustration of the linearized path co-registered with the lumen diameter and cross sectional area information measured near a stenosis.

FIG. 17 shows a display of position of catheter on linear map.

FIG. 18 shows a block diagram enlisting various modules along with the output it provides to the end user.

FIG. 19 shows variation of SSD with respect to time (or frames).

FIG. 20 shows a histogram of SSD.

FIG. 21 shows a capture image.

FIG. 22 shows a directional tube-likeliness metric overlaid on an original image (shown at 5× magnification).

FIG. 23 show consecutive frames during an X-ray angiography procedure captured at the time of linear translation of a C-arm machine.

FIG. 24 shows a variation of SSD for various possible values of translation.

FIG. 25 shows a detected guidewire.

FIG. 26 shows an example of a self-loop in a guidewire.

FIG. 27 shows a block diagram of a marker detection algorithm.

FIG. 28 shows a block diagram of a linearization algorithm.

FIG. 29 shows an illustration of the 5 degrees of freedom of a C-arm machine.

FIG. 30 show illustrations of the process of highlighting a blood vessel through injecting a dye.

FIG. 31 shows the skeletonization of the blood vessel path.

FIG. 32 shows a block diagram of an automatic QCA algorithm.

FIG. 33 shows a block diagram of a fly-through view generation algorithm.

FIG. 34 shows a block diagram of various algorithms involved in the analysis mode of operation.

### DETAILED DESCRIPTION OF THE INVENTION

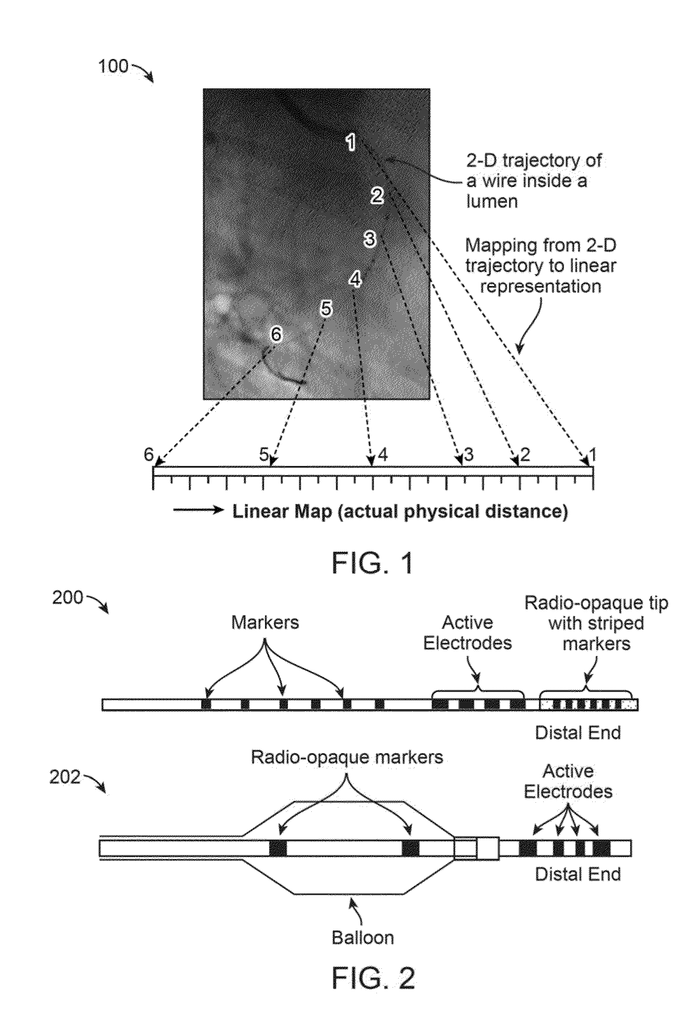

Here we describe methods to process the 2-D images to arrive at a linearized representation of a lumen of a moving organ. Illustrations of the proposed methods are shown for intervention of the coronary artery. A linear map is a mapping from a point on the curved trajectory of a lumen (or the wire inserted into the lumen) to actual linear distance measured from a reference point. This is shown in the schematic 100 in FIG. 1.

Note that in some sections of the blood vessel, the actual lumen trajectory in 3-D may be curving into the image (i.e. it subtends an angle to the viewing place). In these cases, an apparently small section of the lumen in the 2-D curved trajectory may map to a large length in the linear map. The linear map represents the actual physical distance traversed along the longitudinal axis of the lumen when an object traverses through the lumen.

The linear mapping method is applicable in a procedure using any one of the following endo-lumen instruments in a traditional (2-D) coronary angiography:
1. Guidewire with active electrodes that are radio-opaque and/or markers as disclosed in hereinabove.
2. Catheter with electrodes that are radio-opaque as disclosed hereinabove used with a standard guidewire
3. A standard guidewire used with a standard angioplasty, pre-dilatation or stent delivery catheter containing radiopaque markers.
4. Any catheter (IVUS, OCT, EP catheters), guidewire, or other endo lumen devices that have at least one radiopaque element (that can be identified in the X-ray image).

Apart from the above mentioned devices, a similar approach can also be used for obtaining a linear map in coronary computed tomography (3-D) angiographic images and bi-plane angiographic images, using only a standard guidewire. The linear map generation can later be used for guiding further cardiac intervention in real-time during treatment planning, stenting as well as pre- and post-dilatation. It can also be used for co-registration of lumen cross-sectional area measurement measured either with the help of QCA or using multi-frequency electrical excitation or by any other imaging (IVUS,OCT, NIR) or lumen parameter measurement device where the parameters need to be co-registered with the X-ray. Standard guidewire and catheter as well as guidewire and catheter with added electrodes and/or markers are referred to as an endo-lumen device in the rest of the document.

Construction Guidewire and Catheter with Markers and/or Electrodes

FIG. 2 illustrates the construction of a guidewire 200 and catheter 202 with active electrodes and markers as shown. The spacing and sizes are not necessarily uniform. The markers and electrodes are optional components. For example, in some embodiments, only the active electrodes may be included. In other embodiments, only the markers or a subset of markers may be included. If the guidewire 200 has no active electrodes or markers, it is similar to a standard guidewire. Even without the markers or electrodes, the guidewire is still visible in an X-ray image. The coil strip at the distal end of a standard guidewire is made of a material which makes it even more clearly visible in an X-ray image. If the catheter 202 does not have active electrodes, it is similar to a standard balloon catheter, which has a couple of radio-opaque markers (or passive electrodes) inside the balloon.

There are several modifications and variations possible to the illustrated constructions in terms of geometry, locations, number and size of markers/electrodes as well as spacing between them. Apart from using active electrodes for linearizing, the guidewire 200 and catheter 202 may be constructed with multiple radiopaque markers which are not necessarily electrodes. Radiopaque markers in a guidewire are shown in FIG. 2. It can either be placed on the proximal side or distal side of the active electrodes. It can also be placed on both the sides of the active electrodes or could be replace them for the purposes of artery path linearization. If the markers on the proximal side of the electrodes span the entire region from the location of the guide-catheter tip to the point where the guidewire is deep-seated, linearization can be done independently for each phase of the quasi-periodic motion. But such constructions are often not desired during an intervention as it often visually interferes with other devices or regions of interest. Hence a reduced set of markers are often desirable. Apart from these, another configuration of the possible guidewire would be to make the distal coil section of the guidewire striped with alternating strips which are radiopaque and non-radiopaque in nature, of precise lengths which need not necessarily be uniform. These proposed modifications may be used independently or together in any combination for artery path linearization. The distal radiopaque coil section of a standard guidewire (without it being striped) can also be used for getting an approximate estimate of the linearized map of the artery. This estimate becomes more and more accurate as the frame rate of the input video increases. All of these variations are anticipated and within the scope of this invention.

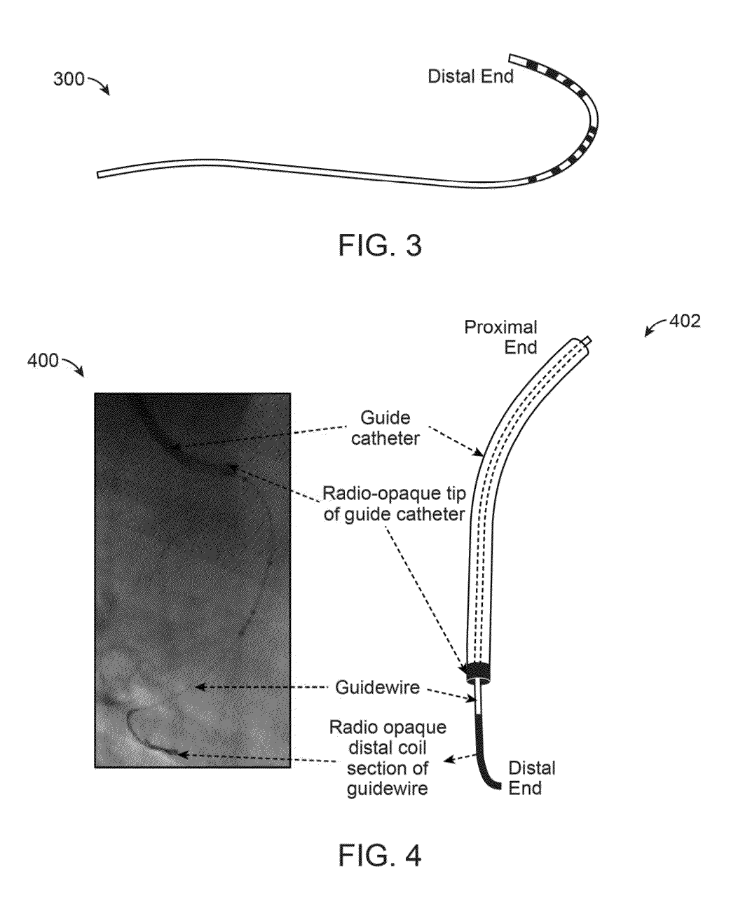

When the endo-lumen device is inserted into an artery, it follows the contours of the artery. When a 2-D snapshot of the wire is taken in this situation, there would be changes in the spacing, sizes and shapes of the electrodes depending on the viewing perspective. For instance, if the wire is bending away from the viewer, the spacing between markers would appear to have reduced. This is depicted by the curved wire 300 shown in FIG. 3.

Description of Various Use Cases

This sub-section describes the various use cases in which the generation of a linearized map would be of clinical significance.

When the linearization is done using guidewire with markers and electrodes, or using standard guidewire, the linearized map can be used for co-registration of anatomic landmarks (such as lesions, branches etc.) with lumen measurement.

Such co-registration can serve several purposes:
1. The points of interest in such as a lesion can be then superimposed back onto an angiographic view
2. Other therapy devices (such as stent catheters, balloon catheters) can be guided to the region of interest
3. Alternatively, the advancement of any device along the co-registered artery can be displayed in the linear view to guide therapy.

If a standard-guide wire is used along with a catheter consisting of markers/electrodes, and the markers or electrodes in catheter is used for linearization during pre-dilatation, computer-aided intervention assistance can be provided for all the further interventions. This holds well even if the linearized map is generated using standard catheter containing radiopaque balloon markers. Once linearized, the artery map which is specific to the patient can also be used for other future interventions for the patient in that artery.

Description of the Algorithm

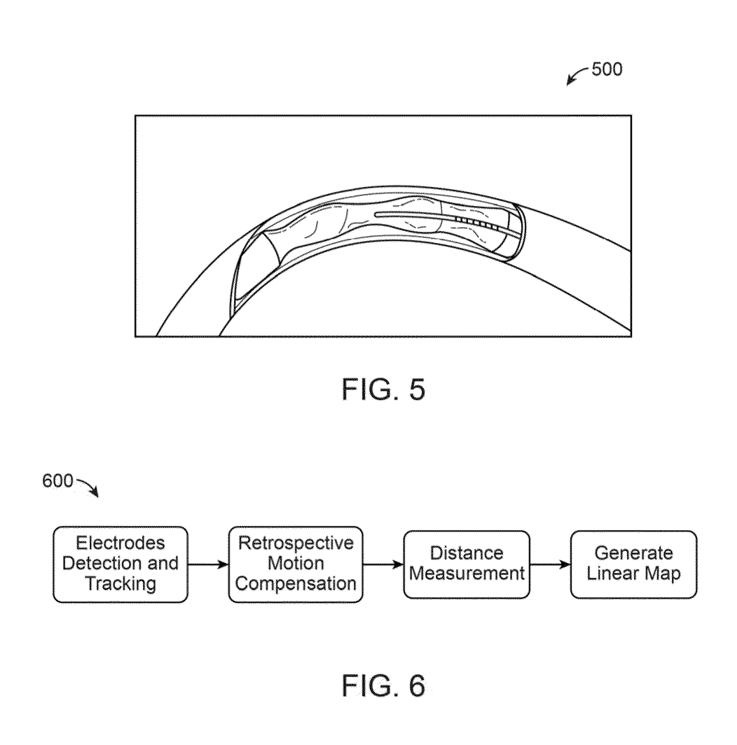

The guidewire, guide catheter and catheter used in an angiographic procedure are shown in the fluoroscopic image 400 of FIG. 4. The guidewire and guide catheter 400 are further shown illustrating how the guidewire may be advanced from the catheter. An illustration of the radiopaque markers 500 on a guidewire inside a coronary artery is shown in FIG. 5.

The algorithm that is described here is for linearization of a lumen with reduced set of markers. Markers spanning the entire length of the artery can be seen as a special case of this scenario. For achieving the goal of artery path linearization, we detect the radiopaque markers (either active electrodes in the guidewire and catheters or balloon markers) in the endo-lumen device and track them across frames through different phases of heart-beat. Retrospective motion compensation algorithm is then used to eliminate the effect of heart-beat and breathing for measuring the distances travelled by the electrodes within the artery. The measured distance in pixels is converted to physical distance (e.g. mm) in order to generate a linearized map of the geometry of the coronary artery. FIG. 6 shows a block diagram 600 of an overview of the steps involved.

The challenges in achieving each of these tasks are described below. The radiopaque nature of the markers makes them quite prominently visible in an angiographic image. Several methods such as edge-detectors, interest-point detectors, template matching, Hough-transform based methods may be used for detecting the electrodes individually. However, maintaining robustness in the presence of other radiopaque objects such as pacemaker leads and coronary artery bypass graft wires etc. is a challenging task.

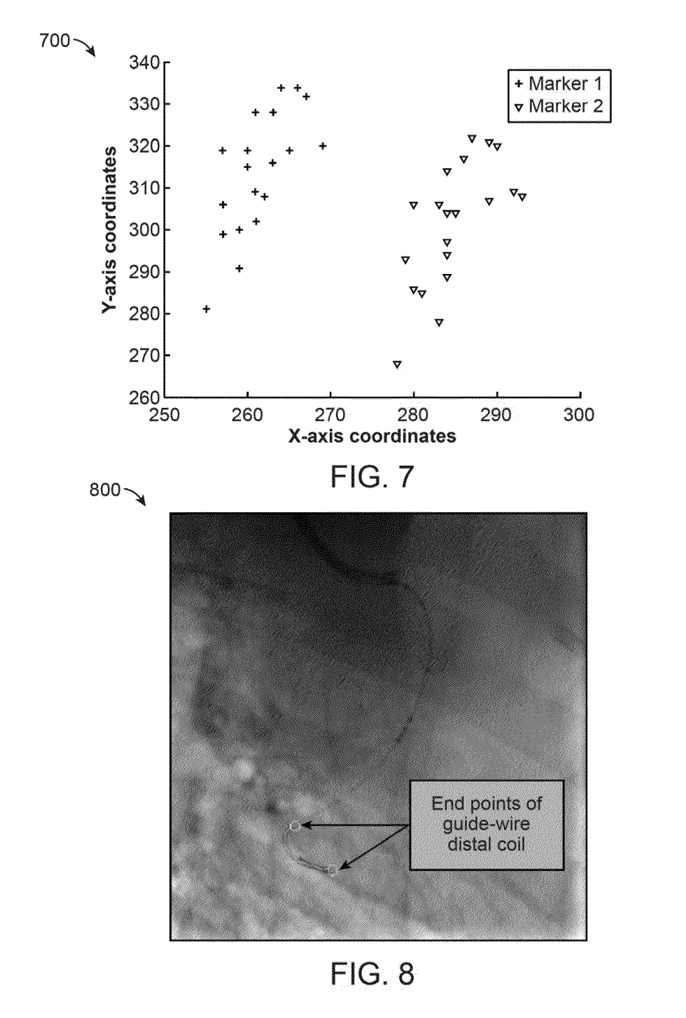

Due to motion observed in an imaged frame, the coordinates of the electrodes in an image need not necessarily remain constant even if the endo-lumen device is kept stationary. It should be noted that the observed motion in an imaged frame could be a result of one or more of the following occurring simultaneously: translation, zoom or rotational changes in the imaging device; motion due to heart-beat and breathing; physical motion of the subject or the table on which the subject is positioned. FIG. 7 illustrates a chart 700 showing the changes in position of two markers in different phases of the heart-beat when the guidewire is stationary.

To compensate for motion of the electrodes, a retrospective motion correction or motion prediction strategy may be used. However, image-based motion correction algorithms are usually computationally expensive and may not be suitable for real-time applications. In our implementation, we segment the image for identifying guidewire. In one embodiment, the entire guidewire is used for motion correction while in another embodiment only a portion of the guidewire in the region of interest is used for motion correction.

In this process, the guidewire is detected in every frame in a manner described later in this section. Markers and electrodes, if any, are also detected in this process. Once the guidewire is robustly detected, known reference points on the guidewire system (guidewire and any catheter it may carry) are matched between adjacent image frames, thereby determining and correcting for motion due to heartbeat between the frames. These reference points may be end points on the guidewire, the tip of the guide catheter, or the distal radio-opaque section of the guidewire, or any marker that has not moved significantly longitudinally due to a manual insertion or retraction of the endo-lumen instrument or any anatomical landmark such as branches in an artery. When the guidewire markers are used for linearization, these markers by definition are not stationary along the longitudinal lumen direction and hence should not be used as land mark points.

Since the trajectory of the catheter is equivalent to that of the guidewire, motion compensation applicable to the guidewire is equally applicable to the catheter. Note that the catheter may actually be moving over the guidewire due to a manual insertion or retraction procedure. Hence the catheter markers should not be used for motion compensation when the catheter is not stationary. In fact, after motion compensation, the movement of the markers on the non-stationary endo lumen device is tracked to determine the position of the device within the lumen.

The segmentation of the guidewire in one frame enables one to narrow down the search region in a subsequent frame. This allows for reduction in search space for localizing the markers as well as making the localization robust in the presence of foreign objects such as pacemaker leads. However, detection of the entire guidewire in itself is a challenging task and the markers are usually the most prominent structures in the guidewire. Hence, our approach considers detecting electrodes and segmenting the guidewire as two-interleaved process. The markers and the guidewire are detected jointly, or iteratively improving the accuracy of the detection and identification, with each iteration, until no further improvement may be achieved.

Motion compensation achieved through guidewire estimation can be used for reducing the amount of computation and the taking into account the real-time need of such an application. However, as mentioned earlier in the section, image-based motion compensation or motion prediction strategy may be used to achieve the same goal by using a dedicated high-speed computation device. The resultant motion compensated data (locations of endo-lumen devices in case of guidewire based motion compensation; image(s) in case of image-based motion compensation) can be used to compute translation of endo-lumen devices/markers along the longitudinal axis of a lumen. This computed information can further be visually presented to the interventionalist as an animation or as series of motion compensated imaged frames with or without endo-lumen devices explicitly marked on it. The location information of the markers and other endo-lumen devices can also be superimposed on a stationary image.

Algorithms for guidewire segmentation as well as algorithms for electrode detection across all the frames are further described in detail herein. Moreover, algorithms for motion compensation through finding the point correspondences between the guidewires in adjacent frames are discussed followed by linear map generation.

Guide Wire Segmentation and Electrode Localization

In one method, our approach for guidewire segmentation comprises four main parts:
1. Reliable detection of the end points of the guidewire.
2. Enhancement of tubular objects in the image.
3. Detection of an optimum path between the two end-points where the optimality is based on continuity of the curve as well as its traversal through the tube-like structures.
4. Localization of the markers in the vicinity of the guidewire and re-estimation of the guidewire segmentation based on the detected markers.

Detection of the End-Points of the Guidewire

Detection of the end-points of the guidewire comprises detecting known substantial objects in the image such as the guide-catheter tip and the radiopaque guidewire strip. These reference objects define the end points of the guidewire. An object localization algorithm (OLA) that is based on pattern matching is used to identify the location of such objects in a frame. In one embodiment of the invention, a user intervenes by manually identifying the tip of the guide catheter by clicking on the image at a location which is on or in the neighborhood of the tip of the guide catheter. This is done in order to train the OLA to detect a particular 2-D projection of the guide-catheter tip. In other embodiments, the tip of the guide catheter is detected without manual intervention. Here, the OLA is programmed to look for shapes similar to the tip of the guide catheter. The OLA can either be trained using samples of the tip of the guide catheter, or the shape parameters could be programmed into the algorithm as parameters. In yet another embodiment, the tip of the guide catheter is detected by analysing the sequence of images as the guide catheter is brought into place. The guide catheter would be the most significant part that is moving in a longitudinal direction through a lumen in the sequence of images. It also has a distinct structure that is easily detected in an image. The moving guide catheter is identified, and the radio opaque tip of the guide catheter is identified as the leading end of the catheter. In yet another embodiment, tip of the guide catheter is detected when the electrodes used in lumen frequency measurement as previously described move out from the guide catheter to blood vessel. The change in impedance measured by the electrodes change drastically and this aids in guide catheter detection. It can also be detected based on injection of dye during an intervention.

The radio-opaque tip of the guide catheter represents a location that marks one end of the guidewire. The tip of the guide catheter needs to be detected in every image frame. Due to the observed motion in the image due to heart-beat, location of the corresponding position in different frames varies significantly. Intensity correlation based template matching approach is used to detect the structure which is most similar to the trained guide-catheter tip, in the subsequent frames. The procedure for detecting can also be automated by training an object localization algorithm to localize various 2-D projections of the guide-catheter tip. Both automated and user-interaction based detection can be trained to detect the guide-catheter even when the angle of acquisition through a C-arm machine is changed or the zoom factor (height of the C-arm from the table) is changed. It is assumed throughout the process of linearization that guide catheter tip is physically unmoved. This assumption is periodically verified by computing the distance of the guide catheter tip with all the anatomical landmarks, such as the location of the branches in the blood vessel. When the change is significant even after accounting for motion due to heart-beat, the distance moved is estimated and compensated for in further processing. Locating the branches in the blood vessel of interest is described further herein.

Tip of the guidewire being radiopaque is segmented based on its gray-level values. The radio-opaque tip of the guide catheter represents a location that represents one end of the guidewire section that may be identified.

Once the tip of the guide catheter is identified, the next step is to identify the radiopaque coil strip of the guidewire, which represents the other end of the guidewire that needs to be identified. In some situations, the guide catheter is detected before the guidewire is inserted through the distal end of the guide catheter. In such situations, the radiopaque coil strip at the distal end of the guidewire is detected automatically as it exits out of the guide catheter tip by continuously analyzing a window around the guide catheter tip in every frame. In other situations (other embodiment), the distal radiopaque coil strip of the guidewire is identified by user intervention. The user would be required to select (e.g. though a mouse click) a point that is in the vicinity of the proximal end (the end that is connected to the core of the guidewire) of the guidewire's coil strip. In yet another embodiment, distal end of the guidewire is detected based on its distinctly visible tubular structure and low gray-level intensity.

Since the radiopaque coil strip of the guidewire is strongly visible on an X-ray, it is relatively easy to detect the radiopaque distal end. A gray-level histogram of the image is created. A threshold is automatically selected based on the constructed histogram. Pixels having a value below the selected threshold are marked as potential coil-strip region. The marked pixels are then analysed with respect to connectivity between one another. Islands (a completely connected region) of marked pixels represent potential segmentation results for guidewire coil section. Each of the islands has a characteristic shape (based on the connectivity of the constituting pixels). The potential segmentation regions are reduced by eliminating several regions based on various shape-based criteria such as area, eccentricity, perimeter etc. of the inherent shapes and the list of potential segmentation region is updated. The region which has the highest tube-likeliness metric is selected as the guidewire coil section. Once the coil section is identified, starting from any arbitrary point on the coil section, a search in all the directions is performed to detect the two end points of the coil-strip. The end-point which is closest to that of the corresponding point in the previous frame or from that of the guide catheter tip is selected. This represents the second end point of the guidewire that needs to be identified for guidewire segmentation. The result of detection of the distal coil is shown in the image 800 of FIG. 8. There are 2 end points detected. Of these, the one closer to the point selected by the user is selected in the first image frame.

Due to the observed motion in the image due to heart-beat, location of the corresponding position in different frames varies significantly. Thus the location of guide-catheter tip and the proximal end of the guidewire coil strip changes significantly from frame to frame. To detect the end-points of the guidewire in all the subsequent frames, a region around the detected points in the initial frame is selected. The gray level intensities of the selected regions are considered as a template. A 2-D correlation is performed in a relatively large region around the detected coordinates in the subsequent frames. The location where the correlation score achieves a maximum is selected as the end-points of the guidewire in the sub-Sequent frames. In cases where the global maximum is not ‘significant’ enough, several candidate points are selected. Motion between the previous frame and all the candidate points, distance of the guide catheter point in the current frame to the frame in the same phase, but several previous heart beats is computed. Resultant optimum point minimizes a combination of these distance functions. The algorithm for segmentation of guidewire uses the detected end-points as an initial estimate for rejecting tubular artifacts which structurally resembles a guidewire. Guidewire segmentation procedure also refines the estimate of the position of the end-points.

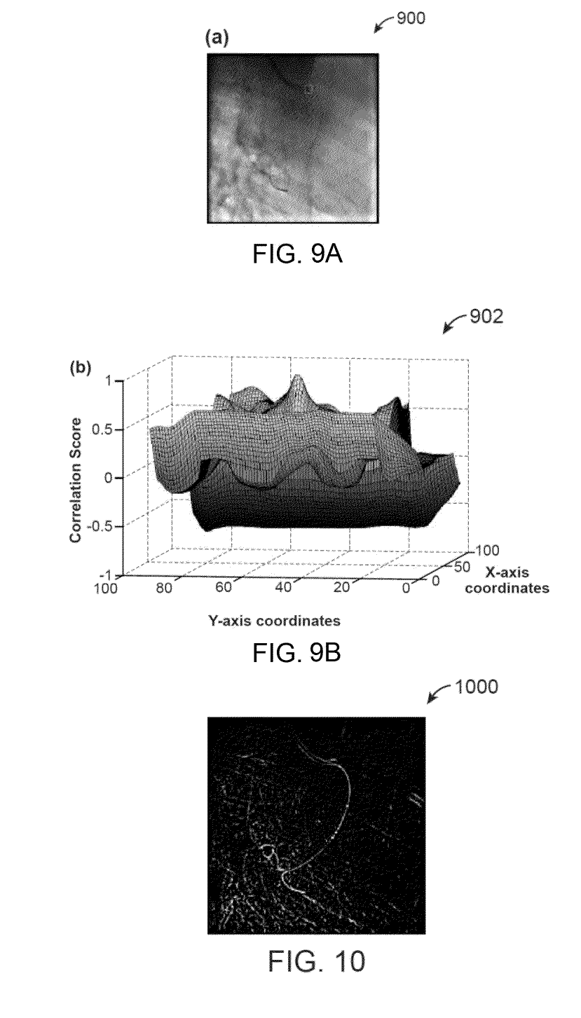

The result of detection of the tip of the guide catheter is shown in FIG. 9A which depicts a localized guide-catheter 900 having a tip based on template matching at one end of the guidewire and a marked tip of the guidewire radiopaque coil at the other end. FIG. 9B shows a chart 902 graphing the variation of the correlation score and the presence of a unique global maximum which is used for localization of the tip of the guide catheter.

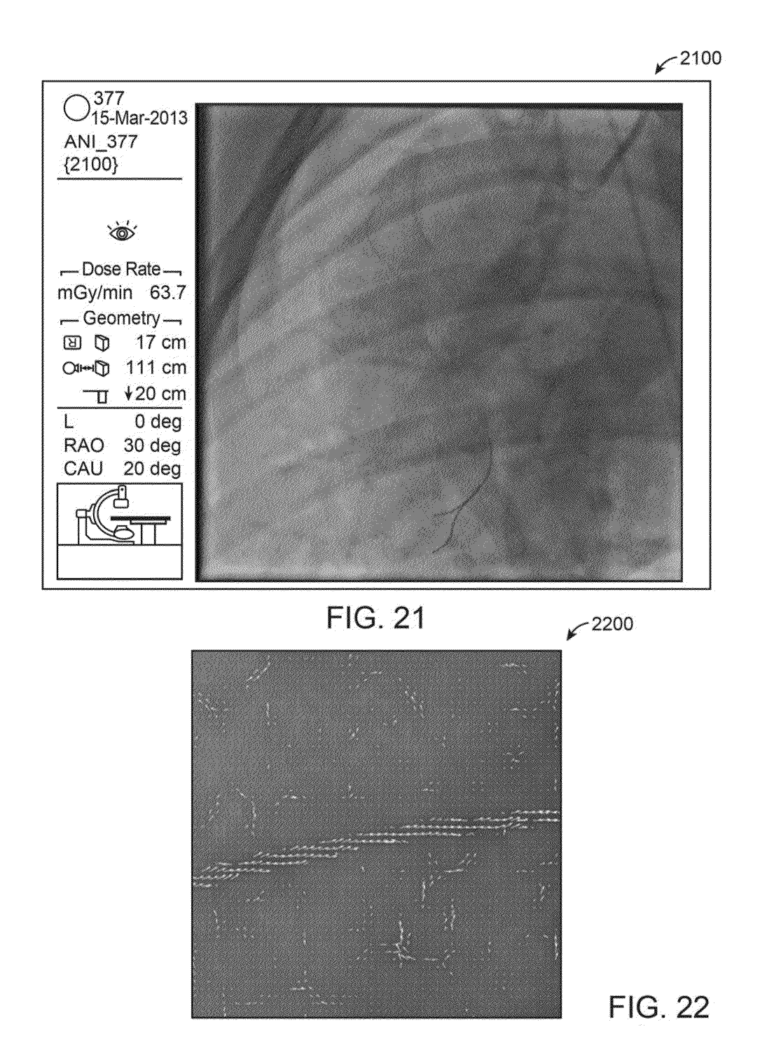

The location of the end-points of the guidewire 900 change significantly when the angle of acquisition through a C-arm machine is changed or the zoom factor (height of the C-arm from the table) is changed. In such situations, the end-point information from the previous frame cannot be used for re-estimation. Either the user may be asked to point at the corresponding locations again or the automatic algorithm designed to detect the end-points without requiring an input from previous frame, as discussed earlier in the section, may be used. The detection of angle change of the C-arm can be done based on any scene-change detection algorithm such as correlation based detection. This is done by measuring the correlation of the present frame with respect to the previous frame. When the correlation goes lesser than a threshold, we can say that the image is considerably different which is caused in turn by angle change. Angle change can also be detected by tracking the angle information available in one of the corners of the live feed images captured (as seen in FIG. 21).

Enhancement of Objects of Interest

Several approaches can be found in the literature for enhancing specific objects of interest. In one embodiment where the objects of interest resemble tube-like structures image enhancement techniques specific to highlighting tube-like structures are used. Some of the commonly used metrics are Frangi's vesselness metric and tube-detection filter. In another embodiment, where the interventional tools do not resemble tube-like structures, image enhancement techniques specific to the geometry of the object of interest is used. To demonstrate implementation, we use Frangi's vesselness measure to enhance the tubular objects in the image. However, any alternative method which serves a similar purpose can be used as its substitute. In Frangi's formulation of tube-likeliness T(x) is defined as:

$$T ( x ) = \{ \begin{matrix}1 & & & if {λ}_{2} > 0 \\ & ( 1 - exp ( - \frac{{s}^{2}}{2 {y}^{2}} ) ) & otherwise & \end{matrix} \} \qquad ( 1 )$$

where λ₁ and λ₂ are eigenvalues of the Hessian matrix of the image under consideration such that ∥λ₁∥≦∥λ₂∥ with S=\sqrt{λ₁²+λ₂²}.

The Hessian matrix is the second order derivative matrix of the image. For each pixel P(x,y) in the image there are four 2^{nd} order derivatives as defined by the 2×2

$$matrix H ( x , y ) = [ \begin{matrix}\frac{{\partial }^{2} P}{\partial {x}^{2}} & \frac{{\partial }^{2} P}{\partial x \partial y} \\ \frac{{\partial }^{2} P}{\partial y \partial x} & \frac{{\partial }^{2} P}{\partial {y}^{2}}\end{matrix} ] .$$

The values α and β are weightage factors and are chosen empirically to yield optimal results.

The result of enhancement 1000 of tubular objects is shown in FIG. 10 (whiter values correspond to pixels that are more likely to be part of a tubular structure; darker values denote lower likelihood). The tube-likeliness metric thus obtained is a directionless metric. For detecting the path of the endo-lumen device, dominant direction of the tube-likeliness metric is sometimes valuable information. For getting the dominant direction information, eigenvector of the Hessian matrix is used. FIG. 22 shows the directional tube-likeliness metric 2200 overlaid on an original image representative of the eigenvector overlaid on the image pixels.

Optimum Path Detection

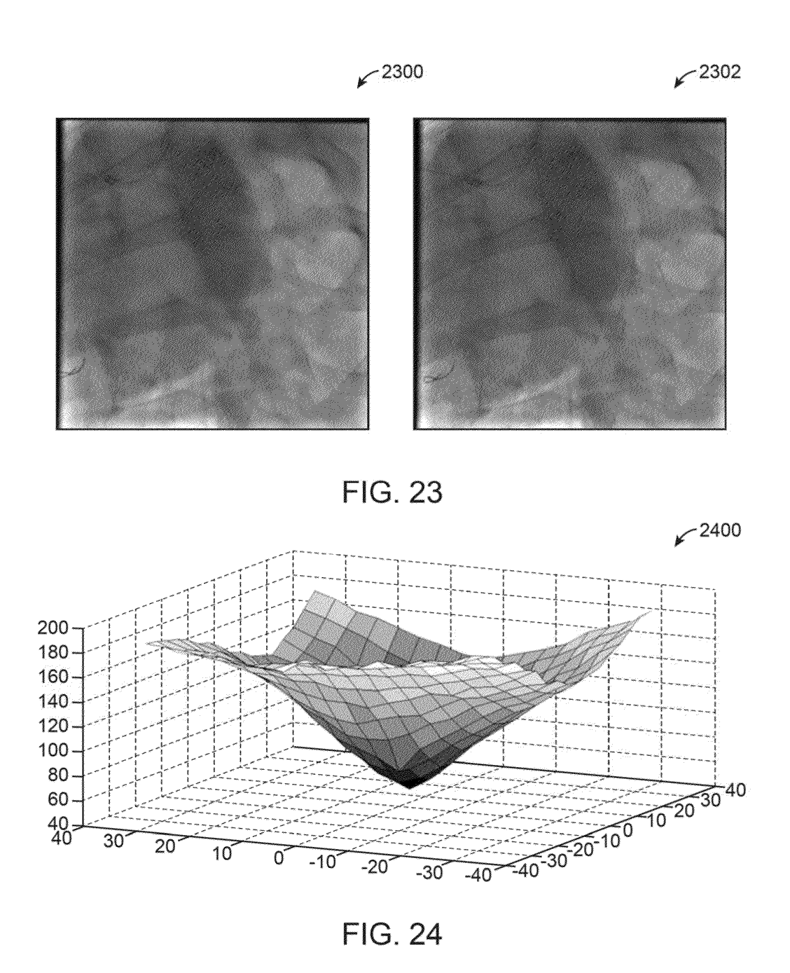

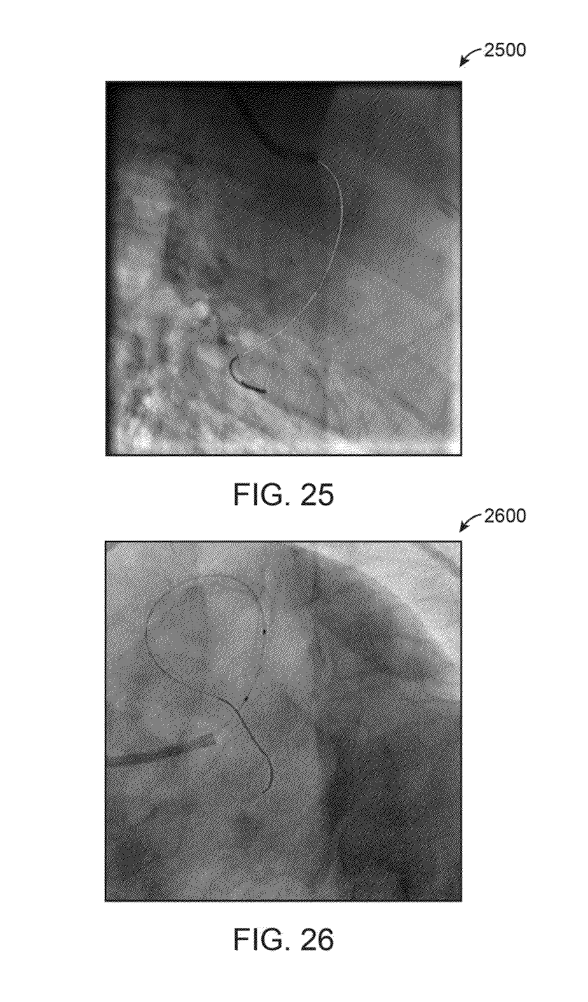

In cases where linear translation of the C-arm position takes place or the zoom factor (height of the C-arm from the table) changes, motion of the same can be estimated. This estimation is based on analyzing the previous frame with respect to the current frame and computing a metric such as sum of squared differences (SSD) or sum of absolute differences (SAD) between the pixels of the images. SSD or SAD is computed for several possible combinations of translation and zoom changes between consecutive frames and the one with minimum SSD/SAD is selected as the correct solution of translation and zoom. For example, FIG. 23 shows 2 consecutive frames 2300, 2302 with slight translation (and no zoom factor change) between the 2 frames 2300, 2302. The SSD values are computed for a wide variety of possible translations varying from −40 to +40 pixels in both the directions. FIG. 24 illustrates a graph 2400 illustrating the variation of SSD values for different possible translations. Minimum is obtained for a translation of 4 pixels in one direction (X-axis) and 12 pixels along the other direction (Y-axis).

C-arm angle changes by a small amount can sometimes be approximated by a combination of translation and zoom changes. Because of this, it becomes essential to differentiate between rotation from translation and zoom changes. While processing a live-feed of images, translation is usually seen seamlessly whereas rotation by an angle, however small that is, causes the live-feed to ‘freeze’ for some time until the destination angle is reached. Effectively, live-feed video contains transition states of translation as well whereas during rotation, only the initial and final viewing angles are seen. In rare cases where the transition state is available in rotation as well, detection of the angle of C-arm as seen in live-feed video 2100 (lower left corner in FIG. 21) can be used to make this differentiation.

Guidewire Detection

Once the end-points of the guidewire are known as well as the tube-likeliness is computed for each pixel in the image, delineation of guidewire reduces to a graph-theoretic shortest-path problem with non-negative weights. More specifically, considering that image pixel in the image is a node, an edge connecting 2 pixels is a vertex, and weight of each vertex is inversely proportional to the tube-likeliness at that point, the guidewire segmentation algorithm can be rephrased as finding a path with the least path-distance. Since the weights under consideration are non-negative, Dijkstra's algorithm or live-wire segmentation which is very well known in the field of computer vision may be used for this purpose.

Alternatively, the segmentation problem may also be viewed as edge-linking between the guidewire end points using the partially-detected guidewire edges and tube-likeliness. Active shape models, active contours or gradient vector flow may also be used for obtaining a similar output.

In our implementation, we use a modified version of Dijkstra's algorithm for segmenting and tracking the guidewire. The Dijkstra's algorithm implemented takes care of the smoothness of the curve being detected by giving some weighting to the previous pixels in the path from the starting point to the pixel under consideration. The search for optimum path is stopped when both the end points (as detected in initialization step) are processed. FIG. 25 highlights the detected guidewire 2500 by such an algorithm. This algorithm can also be used for tracking multiple endo-lumen devices inserted in different blood vessels simultaneously. Alternately, a regular Dijkstra's algorithm can be used to detect and track the guidewires and after they are detected, a separate smoothing function can be applied to obtain a smooth guide-wire.

In several practical scenarios, the guide-catheter tip and the guide-wire tip may go in and out of the frame due to heart beat. In such a case (where at least one of the end point is visible), modified Dijkstra's algorithm is started from one of the end point. Since one of the end-points is out of the frame, the pseudo end point for the optimum path detection algorithm is one of the border pixels in the image. The search for the optimum path is continued until all the border pixels in the image are processed. The path which is nearest to the previously detected guidewire (in the same phase of the heart beat) is chosen as the optimum path.

There is also a possibility of a section of the guidewire going outside a frame while both the end points are visible. In such a case, modified Dijkstra's algorithm is started from both the end-points assuming that only one end-point is visible (based on the above mentioned strategy). Results of both the end-points are combined and the partially absent guidewire path can be reconstructed assuming the continuity of the guidewire doesn't change in the absent region.

In yet another case where the 2-D projection of the 3-D path of the guidewire forms a self-loop, modified Dijkstra's algorithm is used to detect the path where no loop exists. The point where the change of path of the guidewire is abrupt, a separate region based segmentation technique is used to detect the loop in the guidewire. For example, in our implementation, fast marching based level set algorithm is used to detect the loop in the guidewire. This part of the algorithm is set off only in cases where there is a visible sudden change of guidewire direction. FIG. 26 shows an example of such a use case scenario where a self-loop 2600 is shown formed in the guidewire.

The search space of the Dijkstra's algorithm is also restricted based on the nearness of a pixel to the guidewire that was detected in the same phase in several previous heart-beats. The phase of the heart-beat can be obtained by analyzing the ECG or other measuring parameters that are coordinated with the heart beat such as pressure, blood flow, Fractional flow reserve, a measure of response to electrical stimulation such as bio-impedance, etc., obtained from the patient.

In our implementation, we have used ECG based detection of phase of the heart-beat. This is done by detecting significant structures in ECG such as onset and end of P-wave and T-wave, maxima of P-wave and T-wave, equal intervals in PQ segment and ST segment, maxima and minima in QRS complex. If a frame being processed corresponds to the time at which there is an onset of P-wave in ECG signal, for restricting the search space of guidewire detection, frames from several previous onset of P-wave is selected and their corresponding guidewire detection results are used. Frames corresponding to the same phase of the heart-beat need not always correspond to similar shapes of the guidewire. This is due to the fact that apart from motion due to heart-beat, there is also an effect of breathing of the subject that is seen in the video. The motion due to breathing is usually quite slow compared to that of motion due to heart beat. For this reason, an image processing based verification is done on the selected frames. All the frames in which the geographical location of the guidewire (after aligning the end points detected during initialization) correspond to significantly high tube-likeliness metric in the current frame is selected as a valid frame for search space reduction and other frames (which belong to the same phase of the heart beat but does not pass the tube-likeliness criterion—referred to as ‘invalid’ frames in the remainder of the paragraph) are discarded. In another embodiment, compensation for breathing is done for all ‘invalid’ frames as defined above. Mean of detected guidewires in the ‘valid’ frames is computed and is marked as reference guidewire. Point correspondence between the detected guidewires in the ‘invalid’ frames and the reference guidewire is computed as explained further herein. This point correspondence in effect nullifies the motion due to breathing in several phases of the heartbeat. Since this process separates the motion due to heart beat from motion due to breathing, it can be used further to study the breathing pattern of the subject:
1. The information of guidewire's location and shape in the previous frame allows us to narrow down the search range of the guidewire in the current frame.
2. The tube-likeliness metrics computed in the current frame for pixels in the region of interest
3. The guidewire end points in the current frame that is detected based on the guide catheter tip and the radio opaque distal part of the guidewire

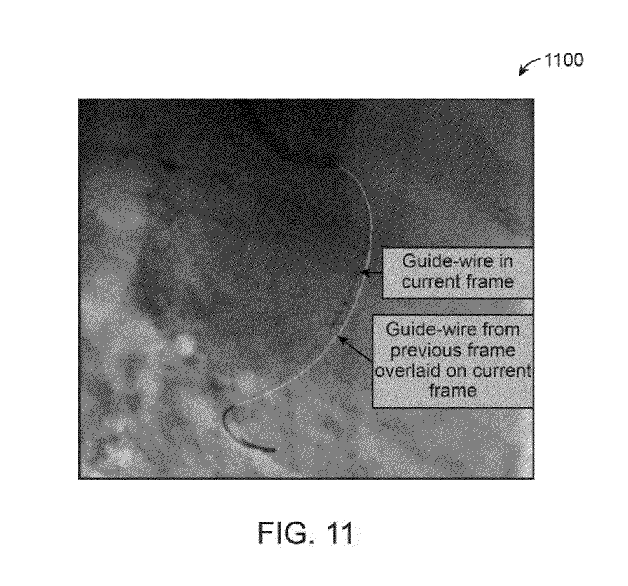

To narrow the search range for detecting the guidewire, the detected guidewire in the previous frame is mapped on to the current frame. Since the end points of the guidewire are known for the present frame, the previous frames guidewire is rotated scaled and translated (RST) so that the end-points coincide. Thus aligned image 1100 of the guidewire from the previous frame is mapped on the current frame as shown in FIG. 11.

It can be noted that the search space for the finding the present guidewire reduces tremendously once an initialization from the previous frame is taken into consideration. The prediction of the position of the guidewire can be made even better if the periodic nature of the change in trajectory due to heartbeat is taken into account. This however is not an essential step and each frame can individually be detected without using any knowledge of the previous frame's guidewire. The detection of guidewire after one complete phase of the heart-beat can consider the guidewire detected in the corresponding phase of the previous cycle of heart beat. Since the heart-beat is periodic and breathing cycles are usually observed at a much lesser frequency, the search-space can be reduced even further. The same phase of the heart-beat can be detected by using an ECG or other vital signals obtained from the patient during the intervention. In cases where vital signs are not available for analysis, image processing techniques can be used for decreasing the search space considerably. Analysis of the path of the endo-lumen device for significant amounts of time shows that the movement is fairly periodic. By selecting frames which have guidewires close to the regions of high tube-likeliness metric in the current frame, there is a high probability of selecting the correct frames for choosing the search space. To a fair extent, the correct frame can also be chosen by prediction filters such as Kalman filtering. This is done by observing the 2-D shape of the guidewire and monitoring the repetition of similar shape of guidewire over time. A combination of these two approaches can be used for more accurate results.

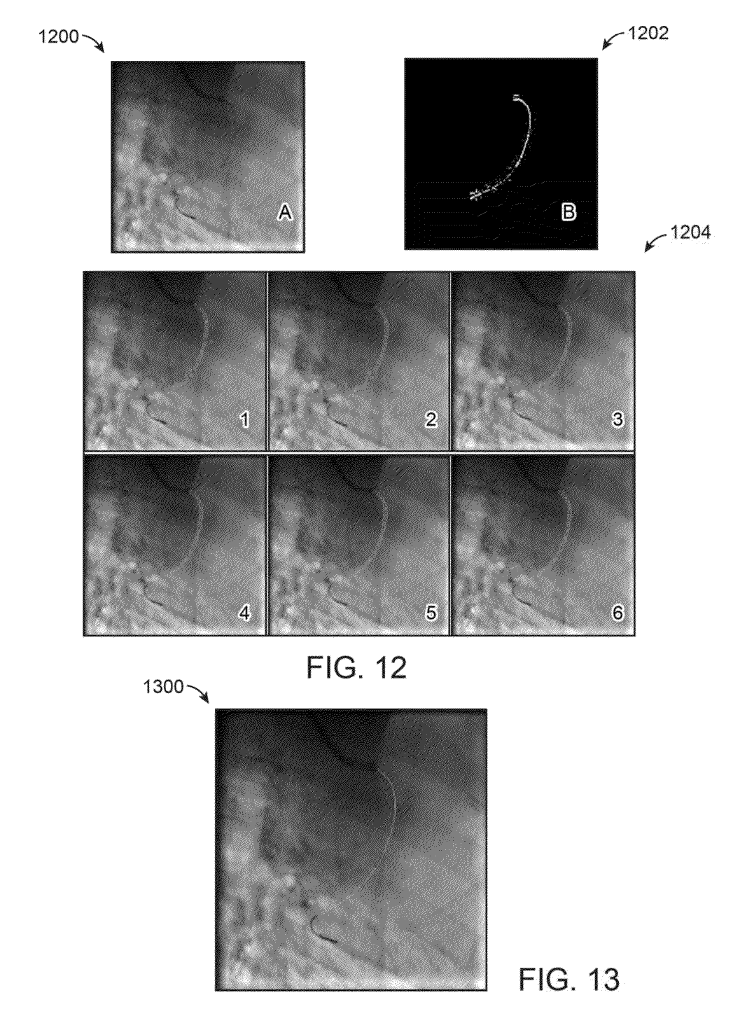

As evident in the FIG. 10, a number of discontinuous edges exist along the actual guidewire. The results of successive refinement of the detected guidewire are shown in the sequence of images shown in FIG. 12. The refinement shown is based on maintaining the continuity of the curve. In this Figure, the image 1200 of FIG. 12(A) is the raw image to be processed. Image 1202 of FIG. 12(B) is the tube Likeliness metric calculated for the image. Images 1204 of FIG. 12 numbered 1 through 6 represent identification of points on the guidewire with successive refinement. The final image (image 6) represents the final identification of points on the guidewire. Cubic spline fitting is then used to delete outliers and fit a smooth curve 1300, as shown in FIG. 13. Direct spline fitting in a noisy data would result in unwanted oscillations. Hence a spline fit with reduced degrees of freedom was used in our implementation.

Radiopaque Marker Detection and Guidewire Re-Estimation

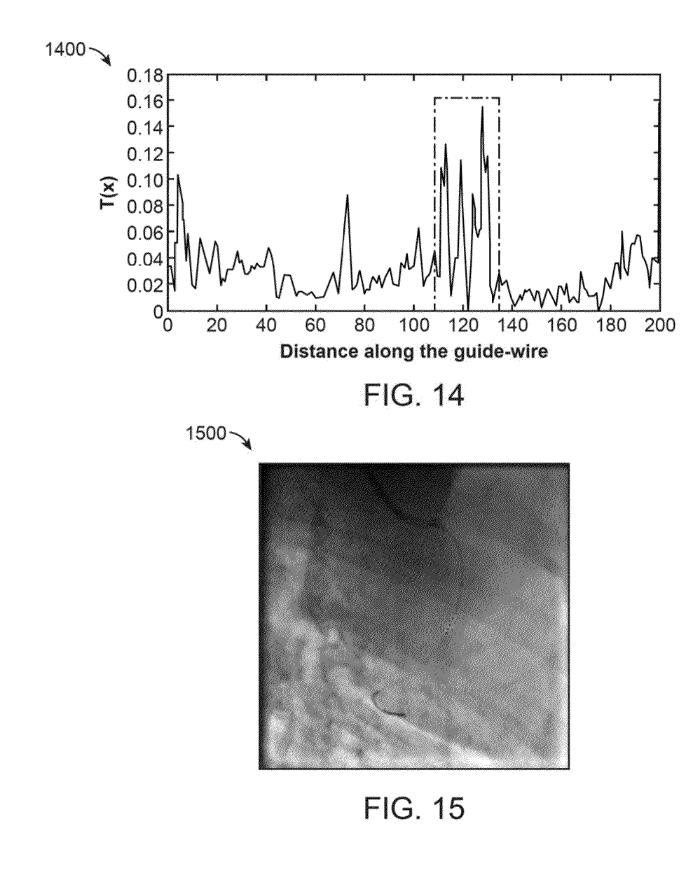

Markers being tubular in nature are often associated with high tube-likeliness metric. Hence, for localizing electrodes, we consider the T(x) values along the guidewire and detect numerous maxima in it. Contextual information can also be used to detect markers. If our aim is to detect balloon markers of known balloon dimensions, say 16 mm long balloon, the search for markers on the detected guidewire can incorporate an approximate distance (in pixel). Thus the detection of markers no longer remains an independent detection of individual markers. Detection of closely placed markers, such as the radiopaque electrodes used for lumen frequency response, can also be done jointly based on the inherent structure of electrodes. FIG. 14 shows a plot 1400 of tube-likeness values of the points on the guidewire. Significant maxima in such a plot usually are potential radiopaque marker locations. This plot 1400 also illustrates the procedure of detecting the inherent structure of the markers under consideration.

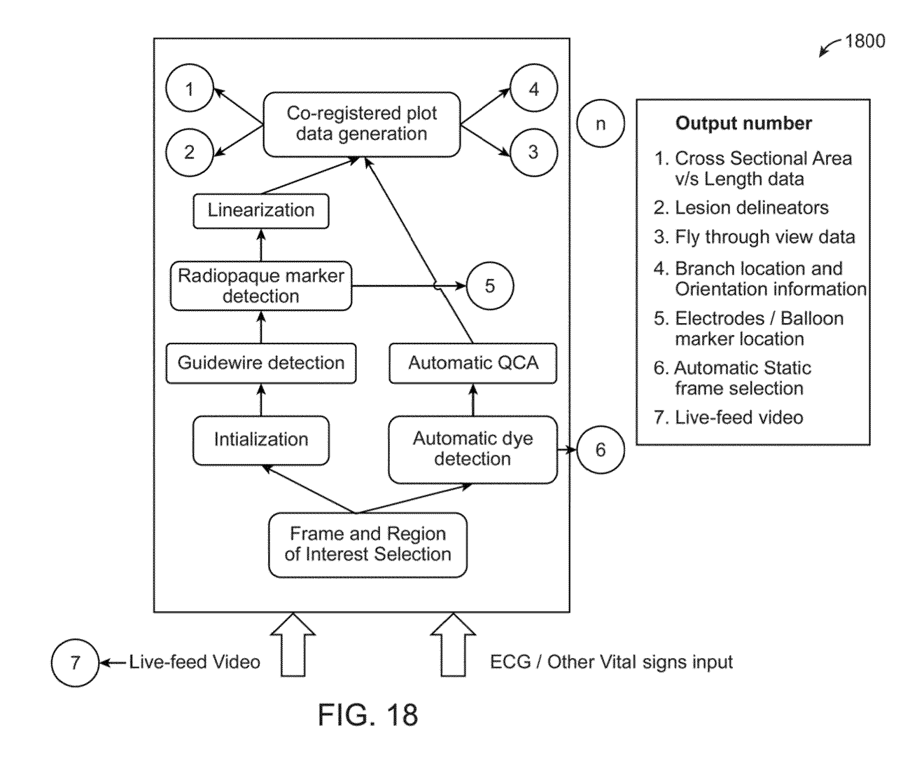

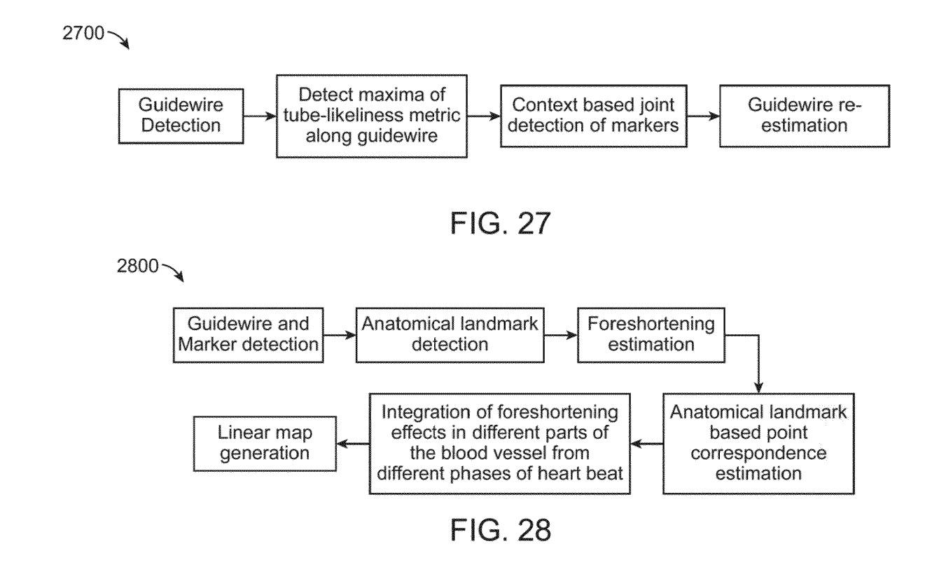

Since the markers are quite prominent structures in an endo-lumen device, the estimated marker locations are considered more reliable, if the detected path of the guidewire does not coincide with the center of the detected markers. In such cases a weighted spline fit algorithm is used to arrive at a better estimate of the guidewire, where markers are given a significantly higher weighting compared to the other points in the guidewire. This is because the markers, having strong features, are more reliably detected than the core of the guidewire. FIG. 15 depicts markers 1500 detected in the image. FIG. 27 shows a block diagram 2700 illustrating the different blocks of the marker detection algorithm. The location of markers is output number 5 as seen in FIG. 18 which illustrates an example of a block diagram enlisting various modules along with the output it provides to the end user.

In the discussion so far, we have assumed that the entire guidewire is visible in the X-ray image. However, in some situations, the guidewire is not clearly visible in the X-ray image. This could be because of poor quality of the X-ray image, low intensity radiation levels being used, or because of the material of the guidewire itself. In these cases, very few points (corresponding to the location of the markers in the endo-lumen device) on the guidewire would show up in the tube likeliness metric map. Guide catheter tip could be used as an additional point of reference. In such situations, only the path between the reliably detected points (markers and guide catheter tip) is estimated using the current frame. Motion compensation algorithm as discussed in the previous section is then applied to the partial guidewire section. As the markers are moved longitudinally along the artery, more segments of the guidewire are estimated. The information of the estimated guidewire segment is propagated both to the subsequent as well as previous frames. This helps in progressively detecting larger segments of the guidewire as the markers are moved and use the information of the trajectory of the markers to build the path of the guidewire. This process would help in building the guidewire path (and thus later create linear map) only till the point the markers progress. But as the markers (active electrodes/balloon markers in catheters) are usually taken at least up to the point where the stenosis occurs, partial generation of linear path would be sufficient for treatment planning and other interventional assistance.

Point Correspondences and Linear Map Generation

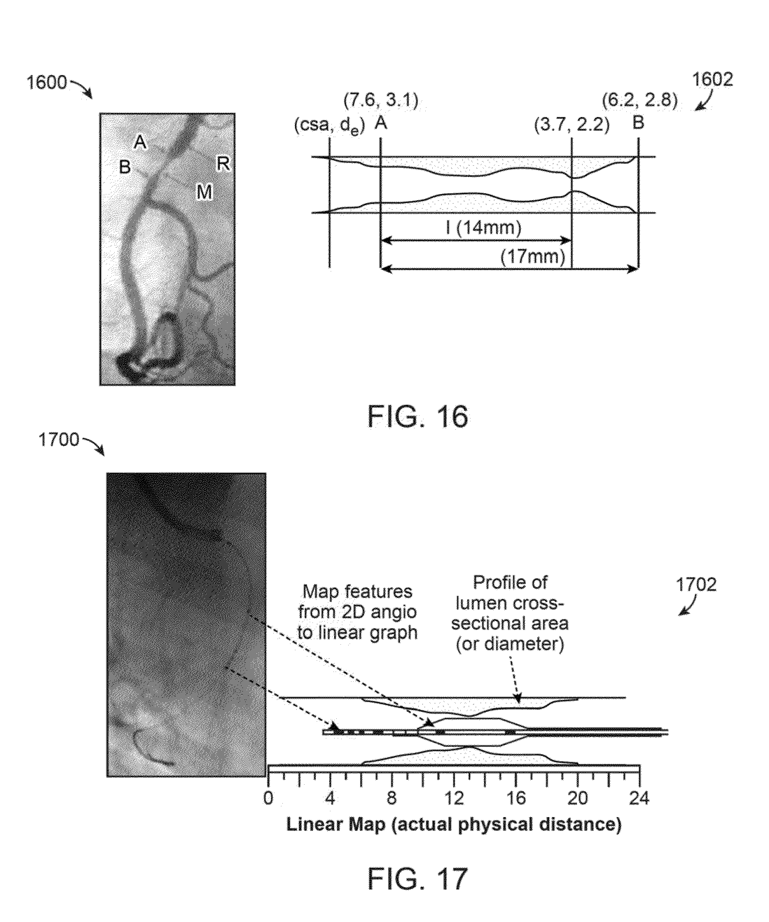

An example of the linear map generation is depicted in FIG. 16 which illustrates the linearized path 1602 co-registered with the lumen diameter and cross sectional area information 1600 measured near a stenosis. After detecting the radiopaque markers on an endo-lumen device, distance between them can be measured along the endo-lumen device (in pixels). Knowing the physical distance between these markers helps in mapping that part of the endo-lumen device into a linear path. If there were closely placed radiopaque markers present throughout the endo-lumen device, a single frame is enough to linearize the entire blood vessel path (covered by the endo-lumen device). The radiopaque markers needs to be placed close enough to assume that path between any two consecutive markers are linear and that the entire endo-lumen device can be approximated as a piecewise linear device.

Note that the mapping between pixels and actual physical distance is not unique. This is because the endo lumen device is not necessarily in the same plane. In different locations, it makes a different angle with the image plane. In some locations it may lie in the image plane. In other locations it may be going into (or coming out of) the image plane. In each case, the mapping from pixels to actual physical distance would be different. For example, if in the former case, the mapping is 3 pixels per millimeter of physical distance, for the latter it could be 2 pixels per millimeter. This physical distance obtained gives an idea of the length of the blood vessel path in that local region.

In an actual use case scenario, placing many radiopaque markers in an endo-lumen device may not be useful for the interventionalist as it might obstruct the view of the path and possible lesions present in them. Thus there is a need to minimize the number of markers placed on the endo-lumen device. The other extreme case is to place a single marker on the endo-lumen device. This would allow us to track the marker in all the frames. If the marker is of known length, the variation of the length of the marker in different locations along the lumen can be used for creating a linearized map. In case a single marker of a significantly small length (where it can no longer be approximated as a line but as a single point on the image) is used, a calibration step of having a motorized pullback is required. This would allow us to map different points in the blood vessel to different points in the linearized map. This will minimize the view obstruction constraint of an interventionalist but at the same time, it adds an additional step (of motorized pullback) for getting the same result. Hence as per our analysis, 2 to 5 closely placed markers near the distal end of the endo-lumen device is an optimum design for aiding an intervention by creating a linearized path. When such an endo-lumen device is inserted, by analyzing multiple frames, as and when the endo-lumen device is pushed in, a linearized view of the blood vessel can be created. It should be noted that for the invention described here, the distance between adjacent markers need not be small enough for the assumption—that they are in the same plane—to hold true. In cases where the distance is large, such as that in balloon markers, distance between corresponding markers in consecutive frames is measured after motion compensation and this distance is further used for linearization.

The observed motion in an imaged frame could be a result of one or more of the following occurring simultaneously: translation, zoom or rotational changes in the imaging device; motion due to heart-beat and breathing; physical motion of the subject or the table on which the subject is positioned. The shape or position of the blood vessel is going to be different in each phase of the aforementioned motion. Thus linearization of the blood vessel is no longer a single solution but a set of solutions which linearizes the blood vessel in all possible configurations of the motion. But such an elaborate solution is not required if the different configurations of the blood vessel is mapped to one another through point-correspondence.

Finding correspondences between corresponding structures has been an extensively researched topic. Image-based point correspondences may be found out based on finding correspondences between salient points or by finding intensity based warping function. Shape based correspondences are often found based on finding a warping function which warps one shape under consideration to another and thus inherently finding a mapping function between each of its points (intrinsic point-correspondence algorithms). Point correspondences in a shape can also be found out extrinsically mapping each point in a shape to a corresponding point in the other shape. This can be either be based on geometrical or anatomical landmarks or based on proximity of a point in a shape to the other when the end points and anatomical landmarks are overlaid on each other. Anatomical landmarks used for this purpose are the branch locations in a blood vessel as described herein. Landmarks that are fixed point on the device or devices visible in the 2-D projection such as tip of the guide catheter, stationary markers, and fixed objects outside the body may also be used. Correlation between vessel diameters (as detected by QCA also described herein) in different phases of the heart beat can also be used as a parameter for obtaining point correspondence. In our implementation, we have used extrinsic point-correspondence algorithm to find corresponding locations of markers in each shape. By finding the point correspondence between different parts of the endo-lumen device in different phases of the heart-beat, foreshortening effect estimated in one phase can be translated to other phase and thus helps in integrating the foreshortening effects. This is used in creating a linearized map of the entire path traversed by the endolumen device. FIG. 28 shows a block diagram 2800 of different blocks involved in the linearization algorithm.

Motion compensation achieved through extrinsic point-correspondence can be used for compensating all of the aforementioned scenarios. It also reduces the amount of computation required for motion compensation as compared to image-based motion compensation techniques. However, as mentioned earlier in the section, image-based motion compensation or motion prediction strategy may be used to achieve the same goal by using a dedicated high-speed computation device. The resultant motion compensated data (locations of endo-lumen devices in case of guidewire based motion compensation; image(s) in case of image-based motion compensation) can be used to compute translation of endo-lumen devices/markers along the longitudinal axis of a lumen. This computed information can further be visually presented to the interventionalist as an animation or as series of motion compensated imaged frames with or without endo-lumen devices explicitly marked on it. The location information of the markers and other endo-lumen devices can also be superimposed on a stationary image.

3-D Reconstruction

Each time a part of the endo-lumen device is linearized, angle it subtends with the 2-D projection plane can be measured based on the apparent foreshortening effect. But there is an ambiguity with respect to whether the part of the endo-lumen device comes out of the plane towards the x-ray receiver or goes away from it. This ambiguity cannot be resolved by this technique. Hence, when linearization of the entire endo-lumen device is done based on ‘n’ separate estimations of foreshortening effect in different parts of the blood lumen trajectory, each part gives a binary ambiguity with respect to 3-D reconstruction of the blood lumen trajectory. The ‘n’ separate estimations may be done based on multiple markers throughout the endo-lumen device or by any sub-sample of it or by any technique mentioned in the above section or by methods mentioned herein. Hence ‘n’ step linearization procedure will have 2ⁿ consistent solutions of 3-D reconstructions. However, not all solutions can be physically possible considering the natural smoothness present in the trajectory of the blood lumen. Several of the 2ⁿ solutions can be discarded based on the smoothness criteria. But a unique 3-D reconstructed path may not necessarily be obtainable based on linearization using a single projection angle alone.

It is a common practice during intervention to view a blood vessel from multiple angles before arriving at a decision. Linearization in multiple angles (at least 2 angles) helps in narrowing down the possibilities of 3-D reconstructed path down to one. This includes, detecting and tracking endo-lumen device and radiopaque markers, motion compensation followed by linearization in at least 2 angles.

In another embodiment, when the projection angle of the C-arm is changed, all possible 3-D reconstructed paths are projected to the new projection angle. Each reconstructed path will have a separate projected path in the new projection angle. Endo-lumen device is detected in the new angle too and all the predicted projections which do not match the detected endo-lumen device's path are rejected. By using projections in multiple angles, verification and narrowing down of 3-D reconstructed path can be done. This procedure helps in finding a 3-D reconstructed path of the blood lumen trajectory.

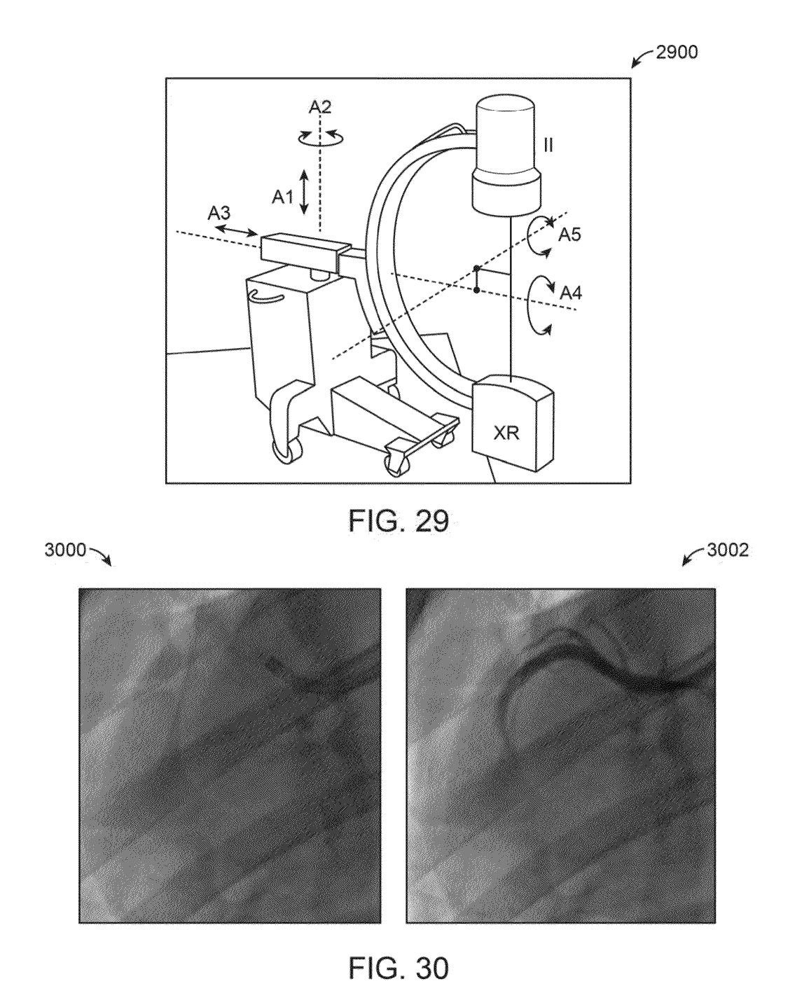

For obtaining a 3-D reconstructed view of the trajectory, the projection angle of the C-arm must be uniquely determined. The C-arm has 6 degrees of freedom. 3 rotational degrees of freedom and 1 translational and 1 magnifying factor (zoom factor). FIG. 29 illustrates the 5 degrees of freedom of a C-arm machine 2900. Uniquely determining each of the 5 parameters is required for accurate 3-D reconstruction. Translation and zoom factors can be obtained by the method explained herein where rotational degrees of freedom can be uniquely determined by analyzing the angle information from the live-feed video data (as seen in FIG. 21). Alternately, it can also be measured using optical or magnetic sensors to track the motion of C-arm 2900. Information regarding the position of C-arm machine 2900 can also be obtained from within the motors attached to it, if one had access to the electrical signals sent to the motors.

Guidance Mode of Operation

Assuming that a co-registered and linearized map already exists, guidance mode of operation helps in guiding treatment devices to the lesion location. In one embodiment, images during the guidance mode of operation are in the same C-arm projection angle as it was at the time of linearized map creation. In such a case, mapping from image coordinates to linearized map coordinates is trivial and it involves marker detection and motion compensation techniques as discussed in previous sections. In another embodiment, the change in projection angle is significant. In such a case, a 3-D reconstructed view of the vessel path is used to map the linearized map generated from the previous angle to the present angle. After transformation, all the steps involved in the previous embodiment are used in this one as well. In yet another embodiment, guidance mode of operation when an accurate 3-D reconstruction is unavailable is done with the help of markers present in the treatment device. In such a case, these markers are used for linearizing the vessel in the new projection angle. Linearizing in the new angle automatically co-registers the map with the previously generated linearized map and thus the treatment device can be guided accurately to the lesion. An example of mapping the position of a catheter 1700 with electrodes and balloon markers is shown positioned along the linear map 1702 in FIG. 17.

This display is shown in real time. As the physician inserts or retracts the catheter, image processing algorithms run in real time to identify the reference points on the catheter, and map the position of the catheter in a linear display. The same linear display also shows the lumen profile. In one embodiment, the lumen dimension profile is estimated before the catheter is inserted. In another embodiment, the lumen dimension is measured with the same catheter using the active electrodes at the distal end of the catheter. As the catheter is advanced, the lumen dimension is measured and the profile is created on the fly.

While the disclosed invention is shown to work with X-ray images, the same concepts can be extended to other imaging methods such as MR, PET, SPECT, ultrasound, infrared, endoscopy, etc. in which the some features of the instrument inserted into a lumen are distinctly visible.

Obtaining Live Video Output, ECG and Other Vital Signs from the Medical Imaging Device

FIG. 18 presents a block diagram 1800 of the details of various modules of the invention along with the output provided to the end user. Each of the various modules is described in further detail herein. DICOM (Digital Imaging and Communications in Medicine) is a standard for handling, storing, and transmitting information in medical imaging. But, it is generally available for offline processing. For the system that is proposed in this invention, live video data, as seen on a display device which an interventionalist uses, is required. For this purpose, either the output of the medical imaging device or the signal that comes to the display device is duplicated. The video input to the display device can either be digital or analog. It can be in interlaced composite video format such as NTSC, PAL, progressive composite video, one of the several variations/resolutions supported by VGA (such as VGA, Super VGA, WUXGA, WQXGA, QXGA), DVI, interlaced or progressive component video etc. or it can be a proprietary one. If the video format is a standard one, it can be sent through a wide variety of connectors such as BNC, RCA, VGA, DVI, s-video etc. In such a case, a video splitter is connected to the connector. One output of the splitter is connected to the display device as before whereas the other output is used for further processing. In cases where the video out is in proprietary format, a dedicated external camera is set up to capture the output of the display device and output of which is sent using one of the aforementioned type of connectors. Frame-grabber hardware is then used to capture the output of either the camera or the second output of video splitter as a series of images. Frame grabber captures the video input, digitizes it (if required) and sends the digital version of the data to a computer through one of the ports available on it such as—USB, Ethernet, serial port etc.

Time interval between two successive frames during image capture (and thus the frame rate of the video) using a medical imaging device need not necessarily be the same as the one that is sent for display. For e.g. some of the C-arm machines used in catheter labs for cardiac intervention has the capability of acquiring images at 15 and 30 frames per second, but the frame rate of the video available at the VGA output can be as high as 75 Hz. In such a case, it is not only unnecessary but also inefficient to send all the frames to a computer for further processing. Duplicate frame detection can be done either on the analog video signal (if available) or a digitized signal.

For duplicate frame detection in the analog domain, comparing the previous frame with the current frame can be done using a delay line. An analog delay line is a network of electrical components connected in series, where each individual element creates a time difference or phase change between its input signal and its output signal. The delay line has to be designed in such a way that it has close to unity gain in frequency band of interest and has a group delay equal to that of duration of a single frame. Once the analog signal is passed through the delay line, it can be compared with the present frame using a comparator. A comparator is a device that compares two signals and switches its output to indicate which is larger. The bipolar output of the comparator can either be sent through a squarer circuit or through a rectifier (to convert it to a unipolar signal) before sending it to an accumulator such as a tank circuit. The tank circuit accumulates the difference output. If the difference between the frames is less than a threshold, it can be marked as a duplicate frame and discarded. If not, it can be digitized and sent to the computer.

In our implementation we have used a digital duplicate frame detector. Previous frame is compared with the present frame by computing sum of squared differences (SSD) between the two frames. Alternately sum of absolute differences (SAD) may also be used. Selection of threshold for selection and rejection of frames has to be adaptive as well. Threshold may be different for different x-ray machines. It may even be different for the same x-ray machines at different points of time. Selecting and rejecting the frames based on a threshold is a 2 class classification problem. Any 2 class classifier may be used for this purpose. In our implementation, we chose to exploit the observation that the histogram of SSD or SAD is typically a bimodal histogram. One mode corresponds to the set of original frames. The other mode corresponds to the set of duplicate frames. The selected threshold minimized the ratio of intra-class variance to inter-class variance.

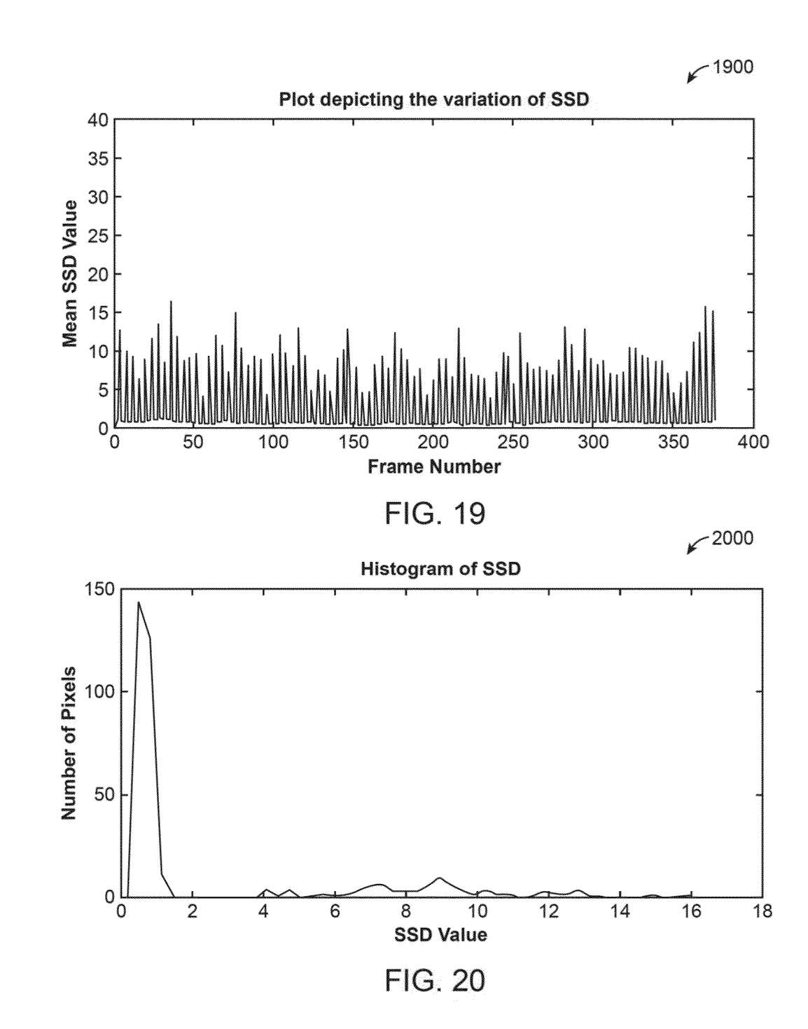

For experimentation purpose, a video with 15 frames per second was displayed at 60 frames per second. FIG. 19 shows a plot 1900 of the variation of mean SSD value computed after digitizing the analog video output of the display device. It can be noted from FIG. 19 that the SSD value has local maxima once in every 4 frames. FIG. 20 illustrates a bimodal histogram 2000 of SSD with a clear gap between the 2 modes.

In our implementation of the proposed system, the video after duplicate frame detection is sent as output from the hardware capture box. This is output number 7 as seen in FIG. 18.

Vital signs on the other hand are easier to tap. ECG out for example typically comes out from a phono-jack connector. This signal is then converted to digital format using an appropriate analog to digital converter and is sent to the processing system.

Automatic Frame and Region of Interest Selection

While processing a live feed of images, not all the frames are useful. An effective data selection algorithm lets you select the images and regions of interest automatically. Unlike DICOM image, live feed data often have several tags embedded on it. For example, FIG. 21 shows a typical live-feed data 2100 captured from a cardiac intervention catherization lab. An intensity based region of interest selection is used to select appropriate region for further processing.

Similarly, during an intervention, the medical imaging device need not necessarily be on at all points of time. In fact, during cardiac intervention using C-arm X-ray machine, radiation is switched on only intermittently. In such a case, the output at the live feed connector is either a blank image, or an extremely noisy image. Automatic frame selection algorithm enables the software to automatically switch between processing the incoming frames for further analysis or dump the frames without any processing.

Tracking of endo lumen device covers initialization, guidewire detection and radiopaque marker detection as mentioned in FIG. 18 and as also disclosed in a number of co-owned patents and patent applications incorporated hereinabove.

Automatic QCA

Automatic QCA is the process of obtaining an approximate estimate of lumen diameter automatically. This is done when a radiopaque dye is injected in the blood vessel. Such a dye highlights the entire blood vessel for a short duration. Important aspects of an automatic QCA algorithm are: detection of the precise moment when the dye is injected through image analysis, selection of the frame where the blood vessel of interest is lighted up completely, finding the skeleton of the blood vessel including all its major branches and then measuring the blood vessel dimensions on either side of the skeleton and thus estimating the diameter in pixels. Knowing the distance between markers in several locations of the artery helps in conversion of the diameter in pixels to millimeter.

Detection of Injection of Dye

The injected dye typically comes to the blood vessel of interest through the guide catheter tip. When the tip is being tracked automatically by an algorithm, presence of dye if gone undetected might result in completely bizarre results for guide catheter detection. FIG. 30 shows in image 3000 a dye being injected into an artery during a cardiac intervention. It can be seen that the characteristic pattern in a guide catheter tip goes completely missing when dye gets injected as shown in image 3002.

For detecting whether a dye is injected or not through image analysis, a region around the guide catheter tip is selected and continuously monitored for sudden drop in the mean gray level intensities. Once the drop is detected, it is confirmed by computing tube likeliness metric around the same region for highlighting large tube like structures. Presence of high values of tube likeliness metric around the region is taken as a confirmation for detecting a dye.

Guide catheter tip provides a good starting point for segmentation of the lighted up vessel as well. In literature, various complex seed-point selection algorithms exist. By tracking guide catheter tip, automatic detection of injection of dye and segmentation of lighted up vessel becomes possible. In theory, a detected guidewire, radiopaque markers, detected lesion, or any significant structure detected in the vessel of interest can be used as seed point for automatic segmentation of the vessel or for automatic injection of dye detection. It can also be detected automatically by connecting a sensor to the instrument used for pumping the fluid in. Such a sensor could transmit signals to indicate that a dye has been injected. Based on the time of transmission of such a signal and by comparing it with time stamp of the received video frames, detection of dye can be done.

Skeleton of Blood Vessel Path

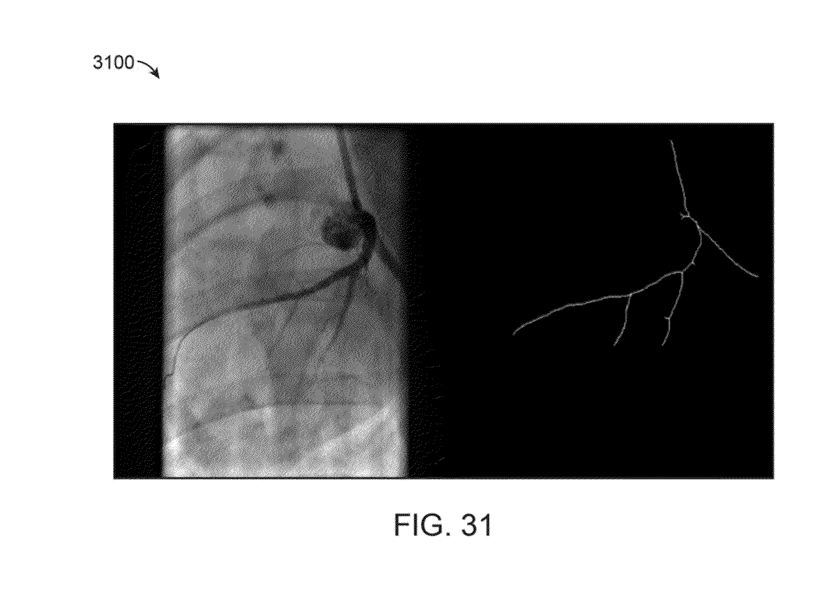

Skeletonization of the artery path, once a dye is injected can be done in multiple ways. Region growing, watershed segmentation followed by morphological operations, vesselness metric based segmentation followed by medial axis transform are some of the algorithms which could be applied. In our implementation, we use vesselness metric to further enhance the regions highlighted by the dye. A simple thresholding based operation is used to convert high tubular valued pixels to whites and the rest to black as seen in the adjacent images 3100 of FIG. 31 which illustrates the skeletonization of the blood vessel path. Selection of the threshold is an important step which enables us to select the regions of interest for further processing. We use an adaptive threshold selection strategy. This is followed by connected component labeling enables the selection of largest island of white pixels, connecting the region near a guide catheter tip. Medial axis transform gives a single pixel wide blood vessel path output. Branches, if any, also get highlighted using this operation. Any point from where a significantly large branch gets separated is detected by analyzing the neighborhood of each point in the detected skeleton. The location of branches is output number 4 as seen in FIG. 18 and it is used as anatomical landmarks to compensate for significant guide catheter movement.

Static Frame Selection

Multiple frames exist where the artery gets lighted up. Selecting the appropriate frame(s) where most of the artery of interest gets highlighted is important as further processing of automatic QCA will be done on the selected image(s). The selected static frame(s) act as a representative of the blood vessel in that particular 2-D projection.

Skeletonization is done for all the frames where a dye is detected. A point on the skeleton is then selected as the most probable location for guide catheter tip. Distance of all the points in the skeleton is computed along the detected path with respect to the estimated guide catheter tip. The point which is farthest from the guide catheter tip, along the direction of the endo-lumen device such as the guidewire (if present) is chosen as the end-point and its corresponding distance is noted. This metric is computed for all the frames. The frame which has largest such metric is chosen as a representative. If no significant maximum exists, multiple such frames are selected. This is output number 6 as seen in FIG. 18.

Blood Vessel Diameter Measurement

On either side of the detected skeleton, a normal is drawn (perpendicular to the direction of the tangent at that location). Along the direction of normal, derivatives of gray level intensities are computed. Points with high values of derivatives on either side of the skeleton are chosen as ‘probable’ candidate points for blood vessel boundaries. For a single point in the skeleton, multiple ‘probable’ points are selected on either side of the contour. A joint optimization algorithm can then be used to make the contour of the detected boundaries pass through maximum possible high probable points without breaking the continuity of the contour. Alternately, only the maximum probability point can be chosen as boundary points and a 2-D smoothing curve-fitting algorithm can also be applied on the detected boundaries so that there are no ‘sudden’ unwanted changes in the detected contours. This is done to get rid of the outliers in the segmentation procedure.

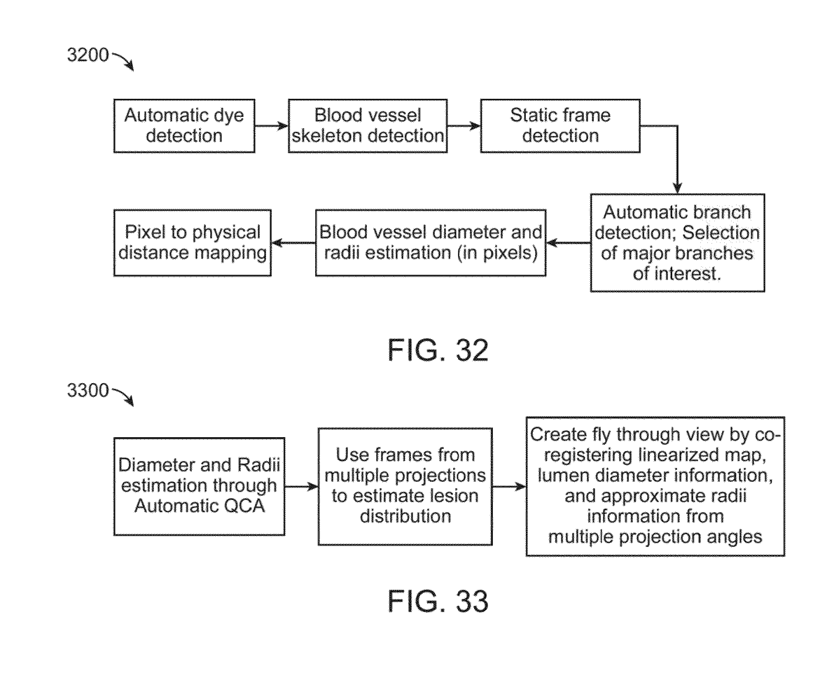

In a normal use case scenario, injected dye progresses gradually within the vessel. Progressively more and more of the vessel gets lighted up in the X-ray. In such a case, a several parts of the vessel may get lighted up in different frames of the video. It is not mandatory for the entire vessel to get lighted up in the same frame. In such a case, the above described joint-optimization algorithm can easily be extended to multiple frames. In cases where similar parts of the artery gets lighted up in multiple frames, joint optimization and estimation will result in more robust estimation of diameter. Similar parts of the artery can be detected using the anatomical landmarks based point-based correspondence algorithm discussed previously herein. Also shown in the block diagram 3200 illustrating an automatic QCA algorithm in FIG. 32.

The distance between 2 corresponding points along the normal of a particular point in the skeleton would give us the diameter of the blood vessel. The difference in the radius along either side of the normal would give us an idea on any abnormally small radius on either side of skeleton. This might in turn aid in detecting which side the lesion is present. Marker positions in different locations along the blood vessel, if present, can be used in aiding the conversion of Automatic QCA results from pixels to millimeter. If these are not present, diameter of the guide catheter tip can be used as a reference for the conversion. The QCA results for blood vessel only serves as an approximate estimate of the diameter since it works on a single 2-D projection. It can act as a good starting point for any lumen diameter estimation algorithms such as OCT, IVUS or the one explained herein. This is output number 1 as seen in FIG. 18.

Lumen diameter estimation when co-registered with a linearized view of the blood vessel would give us an idea regarding the position of a lesion along the longitudinal direction of the blood vessel. However representation of a skewed lesion with the diameter alone can sometimes be misleading. Estimation of left and right radii along the lumen helps in visually representing the co-registered lumen cross-sectional area/diameter data accurately. Alternately, linear scale as generated with the linearization technique can be co-registered on the image with accurately delineated blood vessel to represent QCA and linearized view together.

If Automatic QCA is computed in multiple 2-D projections, it can be combined with the 3-D reconstruction of the blood lumen trajectory (as explained herein). Combination of the two also helps in creating a fly-through view of the blood vessel. Fly through data can also be computed without resolving the ambiguity of 3-D reconstruction (as explained herein). This is output number 3 as seen in FIG. 18 and as also shown in the block diagram 3300 of FIG. 33 which illustrates a fly-through view generation algorithm. The 3-D reconstruction along with lumen diameter information can be used for better visual representation of the vessel and can be used as a diagnostic tool during intervention as well.

Apart from automatic QCA, injection of dye is also quite useful in detecting guide catheter tip automatically as mentioned herein. Since detection of guide-catheter tip is almost a necessity for all further steps in linearization, injection of radiographic fluid whenever angle of the C-arm machine is changed becomes quite useful. If this becomes too much of an overhead for the interventionalist, dye can be injected only in the final view (after the placement of endo-lumen device such as the guidewire), before the placement of stent. This would enable the algorithm to go seamlessly to the ‘guidance’ mode as described herein.

Lesion Delineators

Lesion delineators are the points along the linearized map generated which correspond to medically relevant locations in an image which represent a lesion. Points A and B (as illustrated in FIG. 16) are the points which represent the proximal and distal end of the lesion respectively. The linearized view when co-registered with lumen diameter measurement is capable of detecting this automatically. But the decision of selecting these points interactively during an intervention is left to the judgment of an interventionalist. M is the point of the co-registered plot which correspond the point where the lumen diameter is the least. R is the point on the co-registered plot whose diameter may be taken as a reference for selecting an appropriate stent diameter. The distance between A and B also helps in selecting the appropriate length of the stent. Points A, B, M, and R are collectively known as lesion delineators. This is output number 2 as seen in FIG. 18.

Vessel Compliance and Tortuosity

Tracking the endo-lumen device throughout the procedure and detecting the skeleton of the blood vessel under consideration and all its major branches help in quantifying various biological properties of the blood vessel. Extent of the movement of the artery in different phases of the heart-beat gives us a fair idea about the vessel compliance. By aligning the position of the guide catheter tip and guide wire tip in multiple frames enables us to compare the extent of movement of the blood vessel in different locations. ‘Average’ blood-vessel is worked out by computing the mean location (after alignment at both the ends) of all the points in the detected blood vessel/endo-lumen device. For each phase of the heart-beat, maximum deviation of the blood-vessel path (after aligning at both ends) is computed with respect to the ‘average’ blood vessel. The maximum deviation is inversely proportional to the vessel compliance metric. Vessel compliance gives us an idea of how much a blood vessel's linear representation is valid in different phases of the heart-beat.

Tortuosity of a vessel gives an idea about twists and turns in a vessel. More the tortuosity more is the difficulty in inserting an endo-lumen device such as the guidewire. Moreover, tortuosity of a branch is always more than the tortuosity of its parent branch. Tortuosity is a metric which is directly proportional to the sudden change in direction of an ‘average’ blood vessel.

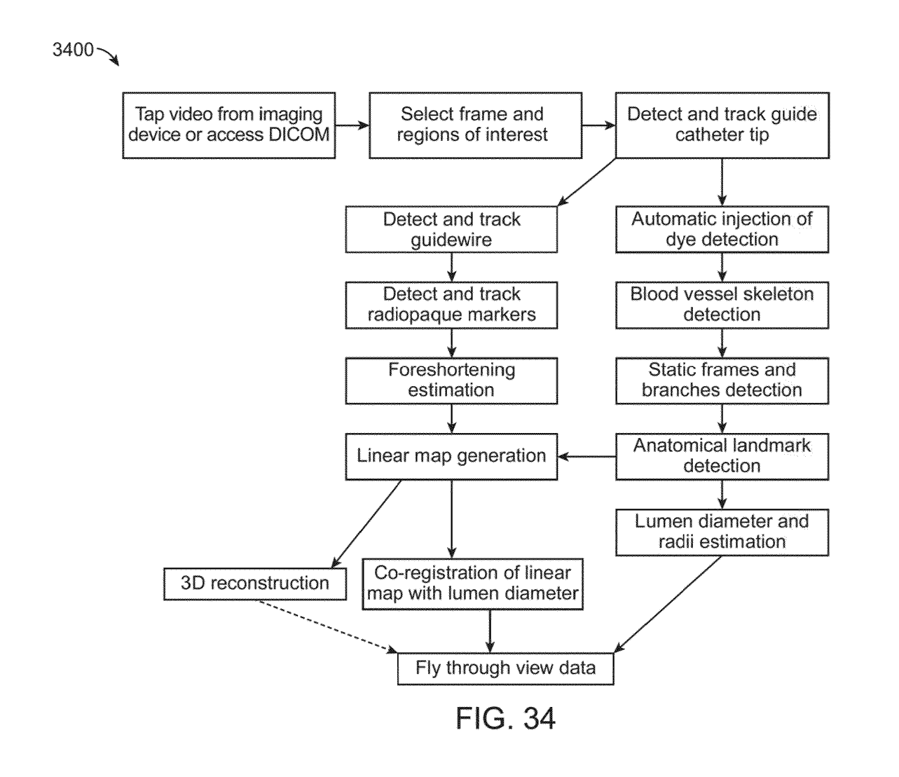

An example of an overall summary is illustrated in the block diagram 3400 of FIG. 34 which illustrates various algorithms described herein involved in the analysis mode of operation.

What is claimed is:

1. A method for generating a linear map from multiple two-dimensional images of a body lumen, comprising:
positioning an elongate instrument having one or more markers within the body lumen to be mapped;
imaging the elongate instrument and the one or more markers along the elongate instrument within the body lumen;
tracking the one or more markers across multiple imaged frames;
matching predetermined reference points along the elongate instrument between the multiple imaged frames; and,
creating a linear map of the body lumen from the multiple imaged frames.

2. The method of claim 1 further comprising enhancing an image for each pixel of the elongate instrument in the multiple imaged frames after imaging the elongate instrument and the one or more markers.

3. The method of claim 1 wherein imaging the elongate instrument comprises moving the elongate instrument through the body lumen while imaging.

4. The method of claim 1 wherein the one or more markers comprise a subset of the region of interest in any single frame.

5. The method of claim 1 further comprising compensating for a motion of the one or more markers due to movement of the body lumen.

6. The method of claim 1 wherein tracking the one or more markers further comprises detecting and tracking the elongate instrument across the multiple imaged frames.

7. The method of claim 1 wherein the elongate instrument comprises a guidewire or catheter.

8. The method of claim 1 wherein the one or more markers comprise electrodes and/or radio-opaque markers.

9. The method of claim 1 wherein the plurality of markers are spaced apart from one another at known distances.

10. The method of claim 1 further comprising injecting a dye into the body lumen during imaging

11. The method of claim 10 wherein injecting a dye into the body lumen comprises automatically detecting the dye within the body lumen.

12. The method of claim 1 further comprising co-registering one or more locations along the linear map with one or more corresponding landmarks.

13. The method of claim 12 wherein the one or more landmarks comprise of any of anatomical landmarks, known positions of parts of the elongate instrument, and geometrical landmarks

14. The method of 13 wherein the anatomical landmarks are determined based on the images that show a dye being injected

15. The method of claim 1 wherein tracking further comprises compensating for the effects of anatomical movement on the elongate instrument relative to the imaged frames.

16. The method of claim 15 wherein compensating comprises segmenting the length of the elongate instrument such that a sub-set of the elongate instrument is used.

17. The method of claim 1 wherein tracking comprises identifying at least one of the visible end points of the elongate instrument.

18. The method of claim 17 wherein identifying end points comprises identifying at least one of a distal tip of the elongate instrument and a guide catheter tip.

19. The method of claim 1 wherein matching predetermined reference points comprises aligning the reference points from each of the multiple imaged frames to coincide.

20. The method of claim 1 wherein matching further comprises determining a distance between adjacent markers from each of the imaged frames.

21. The method of claim 20 wherein determining a distance comprises determining a number of pixels between the adjacent markers.

22. The method of claim 1 wherein matching further comprises determining a distance between corresponding markers in any two imaged frames.

23. The method of 22 wherein determining a distance comprises determining a number of pixels between the corresponding markers in the any two imaged frames

24. The method of claim 21 wherein creating a linear map comprises converting the number of pixels to a physical distance.

25. The method of claim 1 further comprising displaying a position of the elongate instrument upon the linear map.

26. The method of claim 1 wherein the imaged frames are generated via X-ray, MR, PET, SPECT, ultrasound, infrared, or endoscopic imaging.

27. The method of claim 1 further comprising generating a three-dimensional reconstruction of the body lumen.

28. A method for determining the translation of an elongate instrument from multiple two-dimensional images of a moving body lumen, comprising:
positioning an elongate instrument having one or more markers within the body lumen to be mapped;
imaging the elongate instrument and the one or more markers along the elongate instrument within the body lumen;
tracking the one or more markers across multiple imaged frames;
matching predetermined reference points along the elongate instrument between the multiple imaged frames;
compensating for the effect of movement of the body lumen on the elongate instrument: and,
determining the translation of the elongate instrument and one or more markers along the longitudinal axis of the lumen.

29. The method of claim 28 further comprising superimposing the translation of the elongate instrument and one or more markers upon a stationary image of the body lumen

30. The method of claim 28, wherein the movement of body lumen is due to any combination of heartbeat of the subject, breathing of the subject, movement of the subject, change in camera position, and movement of the platform on which the subject is placed

31. The method of claim 28 further comprising creating a linear map of the body lumen from the multiple imaged frames.

32. The method of claim 31 wherein superimposing a translation comprises superimposing the translation of the endoluminal instrument and one or more markers on the stationary image of the body lumen.

33. The method of claim 28 further comprising enhancing an image for each pixel of the elongate instrument in the multiple imaged frames after imaging the elongate instrument and the one or more markers.

34. The method of claim 28 wherein imaging the elongate instrument comprises moving the elongate instrument through the body lumen while imaging.

35. The method of claim 28 wherein the one or more markers comprise a subset of the region of interest in any single frame.

36. The method of claim 28 wherein tracking the one or more markers further comprises detecting and tracking the elongate instrument across the multiple imaged frames.

37. The method of claim 28 wherein the elongate instrument comprises a guidewire or catheter.

38. The method of claim 28 wherein the plurality of markers comprise electrodes and/or radio-opaque markers.

39. The method of claim 28 wherein the plurality of markers are spaced apart from one another at known distances.

40. The method of claim 31 further comprising co-registering one or more locations along the linear map with one or more corresponding landmarks.

41. The method of claim 28 wherein the one or more landmarks comprise of any of anatomical landmarks, known positions of parts of the elongate instrument, and geometrical landmarks

42. The method of claim 28 wherein compensating comprises segmenting the length of the elongate instrument such that a sub-set of the elongate instrument is used.

43. The method of claim 28 wherein tracking comprises identifying at least one of the visible end points of the elongate instrument.

44. The method of claim 43 wherein identifying end points comprises identifying at least one of a distal tip of the elongate instrument and a radiopaque coil strip.

45. The method of claim 28 wherein matching predetermined reference points comprises aligning the reference points from each of the multiple imaged frames to coincide.

46. The method of claim 28 wherein matching further comprises determining a distance between adjacent markers from each of the imaged frames.

47. The method of claim 46 wherein determining a distance comprises determining a number of pixels between the adjacent markers.

48. The method of claim 28 wherein matching further comprises determining a distance between corresponding markers in any two imaged frames

49. The method of claim 48 wherein determining a distance comprises determining a number of pixels between the corresponding markers in the any two motion compensated imaged frames

50. The method of claim 28 further comprising displaying a position of the elongate instrument upon the linear map.

51. The method of claim 2 wherein the imaged frames are generated via X-ray, MR, PET, SPECT, ultrasound, infrared, or endoscopic imaging.

52. The method of claim 28 further comprising injecting a dye into the body lumen during imaging

53. The method of claim 52 wherein injecting a dye into the body lumen comprises automatically detecting the dye within the body lumen.

54. The method of claim 28 further comprising generating a 3-dimensional reconstruction of the body lumen.
